(19) United States
(12) Patent Application Publication
(10) Pub. No.: US 2024/0262774 A1
(43) Pub. Date: Aug. 8, 2024

(54) FLUORINATED ETHERS, THERMAL MANAGEMENT FLUIDS, AND METHODS AND APPARATUSES USING THEM

(71) Applicant: CASTROL LIMITED, Pangbourne, Reading (GB)

(72) Inventors: Sorin Vasile Filip, Sunbury on Thames, Middlesex (GB); Marc John Payne, Sunbury on Thames, Middlesex (GB)

(21) Appl. No.: 18/293,032

(22) PCT Filed: Jul. 29, 2022
(86) PCT No.: PCT/IB2022/057083

§ 371 Date: Jan. 29, 2024

Related U.S. Application Data
(60) Provisional application No. 63/227,791, filed on Jul. 30, 2021.

(51) Int. Cl. C07C 43/17; C07C 41/20; C07C 43/12; C07C 43/192; H01M 10/613; H01M 10/6568

(52) U.S. Cl. CPC C07C 43/17; C07C 41/20; C07C 43/12; C07C 43/126; C07C 43/192; H01M 10/613; H01M 10/6568

(57) ABSTRACT

This disclosure relates generally to fluorinated ether compounds and the synthesis thereof. More particularly, this disclosure relates to fluorinated ether based thermal management fluids suitable for use managing heat in battery systems through direct cooling, such as lithium-ion batteries used in electric vehicles, electric motors, and power electronics, methods of using such thermal management fluids, and systems including such thermal management systems.

### CROSS-REFERENCE TO RELATED APPLICATIONS

This application claims the benefit of priority of U.S. Patent Application No. 63/227,791, filed Jul. 30, 2021, which is hereby incorporated herein by reference in its entirety.

### BACKGROUND OF THE DISCLOSURE

### Field of the Disclosure

This disclosure relates generally to fluorinated ether compounds. More particularly this disclosure relates to fluorinated ether compounds and their use in thermal management fluids, e.g., managing heat in battery-powered electrical vehicles, computing data centers, and high-performance computers. This disclosure also relates to methods of making such compounds and thermal management fluids, methods of using such compounds thermal management fluids, and systems including such compounds and thermal management fluids.

### Technical Background

As current flows through electronic components, including CPUs, GPUs, and even batteries, most will generate heat. Typically, as the amount of current flowing into or out of the component increases, the amount of heat that is generated also increases. If the heat that is generated is not dissipated, the electronic component will rise in temperature. Most electronic components have an effective operating temperature range that allows the component to operate safely and efficiently, and if the component exceeds the maximum operating temperature, the electronic components can become ineffective or even result in thermal runaway and failure. In some cases, after a slight rise in temperature, an electronic component may be able to dissipate heat to its surroundings through a simple heat sink or without any thermal management. In other cases, a more specific thermal management system is needed to dissipate heat that is generated by the electronic component. Larger systems that rely on either larger individual components or a larger number of components, including battery-powered electronic vehicles, computing data centers, and high-performance computers require specific thermal management systems to prevent unwanted and unnecessary thermal failure.

Currently, battery-powered electric vehicles almost exclusively use lithium-ion battery technology. However, lithium-ion batteries generate significant heat during fast and ultra-fast electronic vehicle charging, or high demand discharging. For example, during charging, up to 10% of the inputted power ends up as heat. Lithium-ion batteries are also susceptible to variations in battery temperature. For example, optimal lithium-ion battery operating temperatures are in the range of 10° C. and 35° C. Operation is increasingly inefficient as temperatures rise from 35° C. to 70° C., and, more critically, operation at these temperatures can damage the battery over time. Temperatures over 70° C. present increased risk of thermal runaway. As a result, lithium-ion batteries require specific thermal management systems to regulate their temperatures during vehicle operation. As the fast charging of lithium-ion batteries becomes more common, the need remains for efficient systems for thermal management of the batteries.

Similarly, data centers and high-performance computers are being significantly challenged by an increased demand to effectively manage the heat generated during hardware operation as well as manage the total energy usage of the data center. To track the energy usage of the center, the ratio of how much energy is used by the computing equipment to the amount of energy delivered to the data center (i.e. the power usage effectiveness (PUE)) is measured. As demands on CPU and GPU core performance increase, effective thermal management of these components is critical in order to achieve lower PUE, i.e. close to 1. Accordingly, the need remains for efficient systems for thermal management data centers and high performance computers.

Electronic systems may be cooled directly or indirectly, using thermal management fluids to carry heat away from the component as a cooling fluid or coolant. Electronic vehicle battery thermal management is most effectively assured via direct/immersive fluid thermal management. Direct cooling advantageously allows the thermal management fluid (e.g. dielectric fluid) to come into direct contact with the hot components to carry heat away therefrom. Accordingly, electronic vehicle battery pack design increasingly incorporates direct dielectric fluid management and similar advantageous designs can be incorporated into data centers and high-performance computing centers for “direct-to-chip” cooling.

The most common thermal management fluids are based on mixtures of water with glycol. But because water-based fluids typically conduct electricity, they cannot be used in the direct cooling of electrical components of lithium-ion batteries or computer hardware systems. While indirect cooling allows for water-based coolants to be used, the requirement of electrical shielding can create a bottleneck for heat flow in the cooling process. There exist dielectric thermal management fluids that can be used for direct cooling of electrical components due to their non-electrically-conductive nature. However, the thermal properties of such dielectric thermal management fluids are typically poor in comparison to water-glycol.

Currently, fluorinated hydrocarbon and fluorinated ether-based fluids (e.g. 3M NOVEC range) are being used as thermal management fluids in various prototype electrical vehicles, data centers, high performing computer centers, and bitcoin mining farms. While some of these meet coolant required specifications, most suffer a high global warming potential (GWP) due to their fluorinated structure. In particular, these fluorinated fluids contain molecules with per-fluorinated and partially fluorinated backbones. Furthermore, these fluorinated fluids cannot be effectively formulated with hydrocarbon-based fluids.

Thus, there remains a need for improved dielectric thermal management fluids that are non-conductive, meet coolant specifications, including viscosity, flashpoint, and thermal and electrical conductivity requirements, as well as environmental standards. There also remains a need for dielectric thermal management systems utilizing such fluids, especially those suitable for use in the cooling of lithium-ion batteries and hardware systems.

### SUMMARY OF THE DISCLOSURE

One aspect of the disclosure provides fluorinated ether compounds of formula (I):

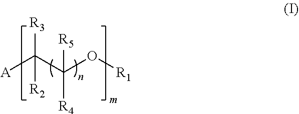

wherein
m is an integer 1, 2, or 3;
n is an integer in the range of 1-16;
A is H or —O—R₆;
R₁ is —C(═CH₂)—CF₃ or —CH(CH₃)—CF₃;
each R₂ and R₃ is independently H or C₁-C₆ alkyl, and each R₄ and R₅ is independently H or C₁-C₆ alkyl, or
each of R₂ and R₄ is H or C₁-C₆ alkyl, and R₃ and R₅ together with the carbons to which they are bound come together to form an C₅-C₇ cycloalkyl; and
R₆ is C₁-C₁₂ alkyl, —C(═CH₂)—CF₃, or —CH(CH₃)—CF₃.

Another aspect of the disclosure provides a thermal management fluid comprising one or more compounds of the disclosure as described herein, the one or more compounds being present in a total amount in the range of 1 wt % to 100 wt % based on the total weight of the thermal management fluid.

Another aspect of the disclosure provides a battery system. The battery system includes a housing; one or more electrochemical cells disposed in the housing; a fluid path extending in the housing and in substantial thermal communication with the one or more electrochemical cells; and a thermal management fluid of the disclosure as described herein disposed in the fluid path.

In another aspect, the disclosure provides an electric vehicle comprising the battery system of the disclosure as described herein.

In another aspect the disclosure provides a thermal management circuit including: a fluid path extending around and/or through a heat source; and a thermal management fluid of the disclosure, disposed in and configured to circulate in the fluid path and to absorb thermal energy produced by the heat source, wherein the fluid is disposed in the fluid path, the heat exchanger, the pump and the connecting duct.

In another aspect the disclosure provides a data center hardware unit comprising the thermal management circuit of the disclosure as described herein. In another aspect the disclosure provides a high performance computer comprising the thermal management circuit of the disclosure as described herein.

Another aspect of the disclosure provides a method including contacting a thermal management fluid of the disclosure with a surface having a temperature of at least 25° C. (e.g., at least 30° C.), the surface being in substantial thermal communication with a heat source; and absorbing thermal energy in the thermal management fluid from the heat source through the surface.

Another aspect of the disclosure provides a method for preparing the compounds of the disclosure. Such method includes contacting a compound of formula (II)

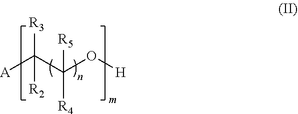

wherein
m is an integer 1, 2, or 3;
n is an integer in the range of 1-16;
A is H, OH, or —O—R₆;
each R₂ and R₃ is independently H or C₁-C₆ alkyl, and each R₄ and R₅ is independently H or C₁-C₆ alkyl, or
each R₂ and R₄ is H or C₁-C₆ alkyl, and R₃ and R₅ together with the carbons to which they are bound come together to form a C₅-C₇ cycloalkyl; and
R₆ is C₁-C₁₂ alkyl;
with CH₂═CF(CF₃) in the presence of a base to provide a first compound of formula (I).

In various embodiments, the method includes hydrogenating the first compound of formula (I) (i.e., in which A is —C(═CH₂)—CF₃) to provide a second compound of formula (I) (i.e., in which A is —CH(CH₃)—CF₃).

### BRIEF DESCRIPTION OF THE DRAWINGS

The accompanying drawings are included to provide a further understanding of the compositions and methods of the disclosure, and are incorporated in and constitute a part of this specification. The drawings are not necessarily to scale, and sizes of various elements may be distorted for clarity. The drawings illustrate one or more embodiment(s) of the disclosure and, together with the description, serve to explain the principles and operation of the disclosure.

FIG. 1 is a schematic cross-sectional view of a thermal management circuit according to an embodiment of the disclosure.

FIG. 2 is a schematic cross-sectional view of a thermal management circuit according to another embodiment of the disclosure.

FIG. 3 is a schematic depiction of a cooling operation of a thermal management fluid of the disclosure.

### DETAILED DESCRIPTION

As described above, the compounds of the disclosure includes one or more compounds of formula (I):

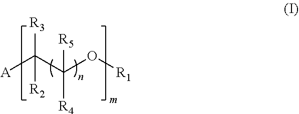

wherein
m is an integer 1, 2, or 3;
n is an integer in the range of 1-16;
A is H or —O—R₆;
R¹ is —C(═CH₂)—CF₃, or —CH(CH₃)—CF₃;
each R₂ and R₃ is independently H or C₁-C₆ alkyl, and each of R₄ and R₅ is independently H or C₁-C₆ alkyl, or
each R₂ and R₄ is H or C₁-C₆ alkyl, and R₃ and R₅ together with the carbons to which they are bound come together to form a C₅-C₇ cycloalkyl; and
R₆ is C₁-C₁₂ alkyl, —C(═CH₂)—CF₃, or —CH(—CH₃)—CF₃.

In the formula above, each of R₁ and R₆ can, in various embodiments, be one of —C(═CH₂)—CF₃ and —CH(CH₃)—CF₃, each connected via ethereal bond to the remainder of the molecule As described below, compounds bearing —C(═CH₂)—CF₃ can be formed by reaction of the corresponding alcohols with CH₂═CF—CF₃. The —C(═CH₂)—CF₃ moiety can be conveniently hydrogenated to provide the corresponding saturated moiety —CH(CH₃)—CF₃.

The compounds of the disclosure can advantageously be provided as polyethers (e.g., diethers, triethers). For example, in various embodiments, in compounds of formula (I) as otherwise described herein R₁ is —C(═CH₂)—CF₃ and A is —O—R₆. In other embodiments, R₁ is —CH(CH₃)—CF₃ and A is —O—R₆.

A wide variety of R₆ groups can be provided. For example, in various embodiments, of the compounds of formula (I) as described herein, R₆ is —C(═CH₂)—CF₃. In other embodiments, R₆—CH(CH₃)—CF₃. In such embodiments, the R₆ group can be the same as or different from the R₁ group, depending on the number and order of etherification reactions and any hydrogenation reactions. For example, in various embodiments as described herein, R₁ is —C(═CH₂)—CF₃, A is —O—R₆, and R₆ is —C(═CH₂)—CF₃. In other embodiments as described herein R₁ is —CH(CH₃)—CF₃, A is —O—R₆, and R₆ is —CH(CH₃)—CF₃. In other embodiments, R₁ is —C(═CH₂)—CF₃, A is —O—R₆, and R₆ is —CH(CH₃)—CF₃. In other embodiments, R₁ is —CH(CH₃)—CF₃, A is —O—R₆, and R₆ is —C(═CH₂)—CF₃.

Of course, a wide variety of other R₆ groups can be provided. For example, in various embodiments as otherwise described herein, in the compounds of formula (I) R₆ is C₁-C₁₂ alkyl. The person of ordinary skill in the art can select the chain length and branching of R₆ based on the disclosure herein to select properties of the overall material, e.g., viscosity and flash point. In various embodiments as otherwise described herein, in the compounds of formula (I) R₆ is C₁-C₁₀ alkyl, such as a C₁-C₈ alkyl, C₁-C₆ alkyl, or C₁-C₄ alkyl. In various embodiments as otherwise described herein, in the compounds of formula (I) R₆ is C₈-C₁₂ alkyl, such as a C₈-C₁₀ alkyl or C₁₀-C₁₂ alkyl. In various embodiments as otherwise described herein, in the compounds of formula (I) R₆ is a branched C₆-C₁₂ alkyl, such as a branched C₆-C₁₀ alkyl, a branched C₆-C₈ alkyl, a branched C₈-C₁₂ alkyl, a branched C₈-C₁₀ alkyl, or a branched C₁₀-C₁₂ alkyl. Branching can be, for example, at an α-position or at a β-position with respect to the oxygen atom to which R₆ is bound. For example, in various such embodiments R₆ is an α-branched C₁-C₁₂ alkyl or is a β-branched C₁-C₁₂ alkyl. In various embodiments, branching is at a β-position with respect to the oxygen atom.

In other embodiments of the compounds of formula (I) as described herein, A is H. For example, in various embodiments of the compounds of formula (I) as described herein, R₁ is —C(═CH₂)—CF₃ and A is H. In other embodiments, R₁ is —CH(CH₃)—CF₃ and A is H.

A variety of R₂, R₃, R₄ and R₅ groups can be provided, selected by the person of ordinary skill in the art based on the disclosure herein to provide the compounds with desired properties, and to take advantage of common olefinic and epoxide feedstocks. For example, in various embodiments of the compounds of formula (I) as described herein, each R₂, R₃, R₄, and R₅ is independently selected from H and C₁-C₄ alkyl (such as C₁-C₃ alkyl or C₁-C₂ alkyl). In various such embodiments, each of R₂, R₃, R₄, and R₅ is independently selected from H and C, alkyl (i.e., methyl).

In various embodiments of the compounds of formula (I), each R₂ is H and each R₃ is H or C₁-C₆ alkyl (such as C₁-C₄ alkyl or C₁-C₂ alkyl); and each R₄ is H and each R₅ is H or C₁-C₆ alkyl (such as C₁-C₄ alkyl or C₁-C₂ alkyl). In various embodiments, each R₂ is C₁-C₆ alkyl (such as C₁-C₄ alkyl or C₁-C₂ alkyl) and each R₃ is C₁-C₆ alkyl (such as C₁-C₄ alkyl or C₁-C₂ alkyl); and each R₄ is H and each R₅ is H. In various embodiments, R₂ is H, R₃ is H, R₄ is H and each R₅ is H or C₁-C₆ alkyl (such as C₁-C₄ alkyl or C₁-C₂ alkyl). In various embodiments, R₂ is H, R₄ is H, R₅ is H and each R₃ is H or C₁-C₆ alkyl (such as C₁-C₄ alkyl or C₁-C₂ alkyl).

In some embodiments, i.e., when n is greater than 1, in each (—CR₂R₃—(CR₄R₅)ₙ—O—) group there will be more than one R₄ group and more than one R₅ group. In some such embodiments, within each such (—CR₂R₃—(CR₄R₅)ₙ—O—) group each R₄ is and only one R₅ group is other than H.)

In various embodiments of the compounds of formula (I), each R₂, R₃, R₄ and R₅ is H.

In various alternative embodiments of the compounds of formula (I), each of R₂ and R₄ is H or C₁-C₄ alkyl (such as C₁-C₃ alkyl or C₁-C₂ alkyl), and R₃ and R₅ together with the carbons to which they are bound come together to form a C₅-C₇ cycloalkyl. In various embodiments as otherwise described herein, in the one or more compounds of formula (I), each of R₂ and R₄ is H or C, (i.e. methyl), and R₃ and R₅ together with the carbons to which they are bound come together to form a C₅-C₇ cycloalkyl. In various embodiments as otherwise described herein, in the one or more compounds of formula (I), each of R₂ and R₄ is H or C, (i.e. methyl), and R₃ and R₅ together with the carbons to which they are bound come together to form a C₆ cycloalkyl.

For example, in various embodiments as otherwise described herein, the disclosure provides a compound of formula (I):

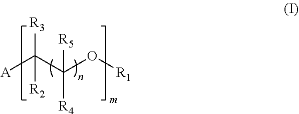

wherein
m is an integer 1, 2, or 3;
n is an integer in the range of 1-16;
A is H or —O—R₆;
R₁ is —C(═CH₂)—CF₃ or —CH(CH₃)—CF₃;
each of R₂ and R₃ is independently H or C₁-C₆ alkyl;
each of R₄ and R₅ is independently H or C₁-C₆ alkyl; and
R₆ is C₁-C₁₂ alkyl, —C(═CH₂)—CF₃, or —CH(CH₃)—CF₃.

And in various alternative embodiments as otherwise described herein, the disclosure provides a compound of formula (I):

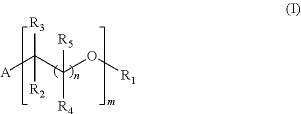

wherein
m is an integer 1, 2, or 3;
n is an integer in the range of 1-16;
A is H or —O—R₆;
R₁ is —C(═CH₂)—CF₃, or —CH(CH₃)—CF₃;
each of R₂ and R₄ is H or C₁-C₆ alkyl, and R₃ and R₅ together with the carbons to which they are bound come together to form a C₅-C₇ cycloalkyl; and
R₆ is C₁-C₁₂ alkyl, —C(═CH₂)—CF₃, or —CH(CH₃)—CF₃;

In various embodiments of the one or more compounds of formula (I) as otherwise described herein, each R₂, R₃, R₄, and R₅ is H, i.e., the compounds have the formula:

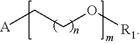

In various embodiments of the one or more compounds of formula (I) as otherwise described herein, each of, R₄, and R₅ is H, and R₃ is H or C₁-C₆ alkyl, i.e., the compounds have the formula

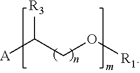

For example, in some embodiments, each of R₂, R₄, and R₅ is H, and R₃ is C₁-C₆ alkyl (such as C₁-C₄ alkyl, C₁-C₃ alkyl, ethyl, or methyl).

The person of ordinary skill in the art can select the chain length n of the optionally repeating unit based on the disclosure herein to select desirable properties of the overall material, e.g., viscosity and flash point. In various embodiments of the compounds of formula (I) as otherwise described herein, n is an integer in the range of 1-12, such as in the range of 1-8. In various embodiments, n is an integer in the range of 1-6, such as in the range 1-4 or in the range of 1-2. In various embodiments, n is an integer in the range of 4-16, such as in the range of 8-16 or in the range of 10-16. In various embodiments, n is an integer in the range of 4-12, such as in the range of 6-12.

In various embodiments, n is an integer 1, i.e., the compounds have the formula:

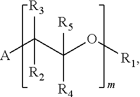

such as

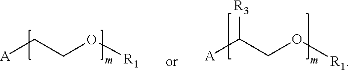

In various embodiments of the compounds of formula (I), A is —O—R₆, n is an integer 1, and each R₂, R₃, R₄ and R₅ is independently H or C₁-C₆ alkyl, i.e. the compounds have the formula:

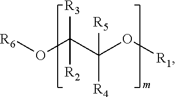

such as

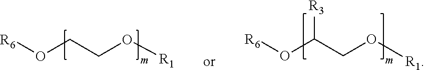

In various embodiments of the compounds of formula I, n is an integer 2, i.e., the compounds have the formula

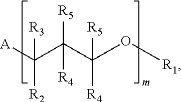

such as

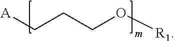

In certain such embodiments, A is —O—R₆, and each R₂ is H and each R₃ is H, and each R₄ and R₅ is independently H or C₁-C₆ alkyl, i.e. the compounds have the formula:

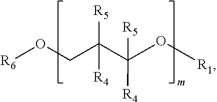

such as

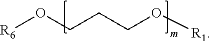

In various embodiments of the one or more compounds of formula (I), m is 1. However, in other embodiments, m is 2, or m is 3.

In various embodiments as otherwise described herein, in the one or more compounds of formula (I) has the formula:

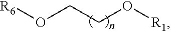

in which n is an integer in the range of 1-16, e.g. in the range of 1-12, or in the range of 1-8, or in the range of 1-4, or in the range of 1-2; R₁ is —C(═CH₂)—CF₃ or —CH(CH₃)—CF₃, and R₆ is C₁-C₁₂ alkyl, —C(═CH₂)—CF₃, or —CH(CH₃)—CF₃. In various such embodiments, R₁ is —C(═CH₂)—CF₃. In various such embodiments, R₁ is —CH(CH₃)—CF₃. In various such embodiments R₆ is methyl or ethyl. In various such embodiments, R₆ is propyl (e.g., n-propyl, isopropyl) or butyl, e.g., n-butyl, t-butyl, sec-butyl or isobutyl. In various such embodiments, R₆ is a branched pentyl, e.g., 1,1-dimethylpropyl, 2,2-dimethylpropyl. In various such embodiments, R₆ is branched, e.g. a group —CH₂—CH(R_{b})(R_{d}) in which R_{b} is methyl or ethyl, and R_{d} is C₃-C₈ alkyl, such as 2-ethylhexyl. In various embodiments as described herein, R₁ is —C(═CH₂)—CF₃ and R₆ is —C(═CH₂)—CF₃. In various embodiments as otherwise described herein R₁ is —CH(CH₃)—CF₃ and R₆ is —CH(CH₃)—CF₃.

The various substituents can be selected to provide the compound with a desirable overall number of carbons, which can effect properties like volatility and viscosity. In various embodiments as otherwise described herein, the one or more compounds of formula (I) contain a total number of carbon atoms from 5 to 30 (e.g., from 5 to 26, from 5 to 22, from 5 to 18, from 6 to 30, from 6 to 26, from 6 to 22, from 8 to 30, from 8 to 26, or from 8 to 22). For example, in various embodiments the one or more compounds of formula (I) contains a total number of carbon atoms from 6 to 22. In various embodiments the one or more compounds of formula (I) contains a total number of carbon atoms from 6 to 20. In various embodiments the one or more compounds of formula (I) contains a total number of carbon atoms from 6 to 18. In various embodiments the one or more compounds of formula (I) contains a total number of carbon atoms from 6 to 16.

Examples of the compounds of formula (I) of the disclosure include, but are not limited to:

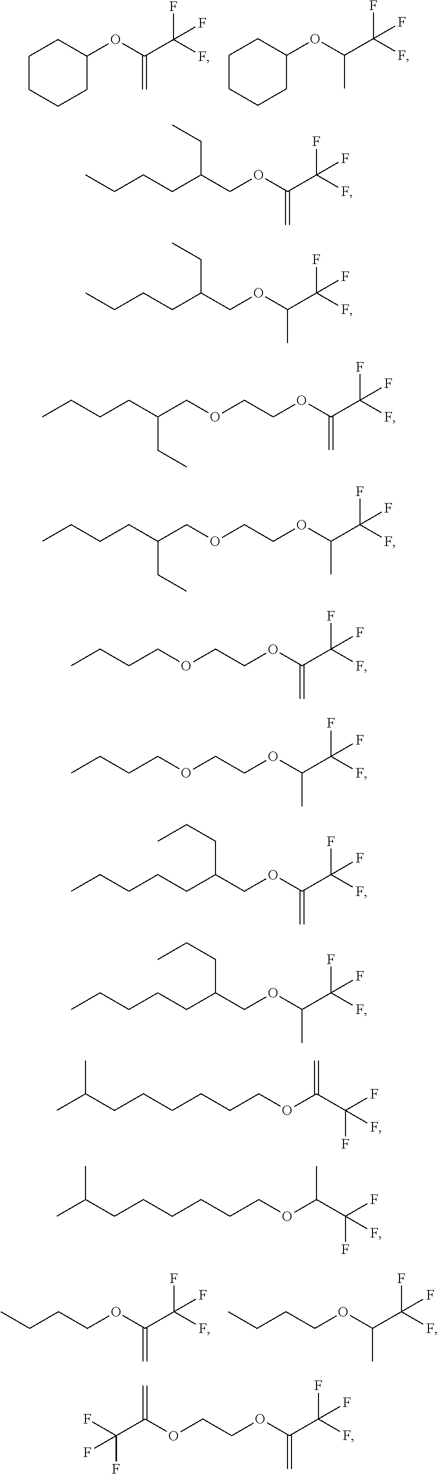

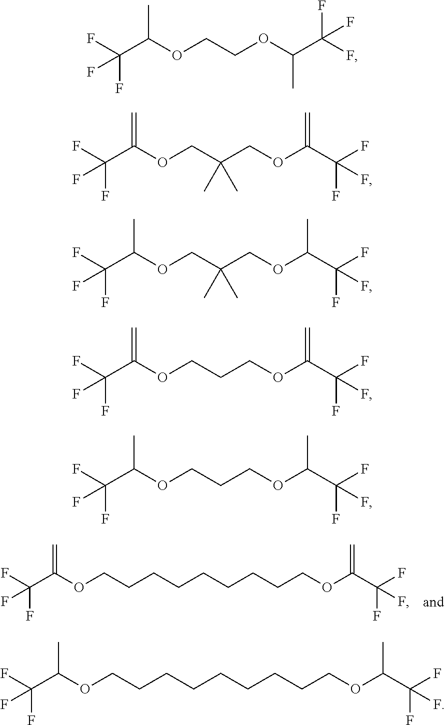

In various embodiments, the compounds can have a flash point of at least 50° C., as measured in accordance with ASTM D93. For example, in various embodiments, a compound as otherwise described herein has a flash point of at least 60° C., e.g., at least 70° C., at least 75° C., at least 80° C., or at least 85° C., measured in accordance with ASTM D93. In various embodiments, a compound as otherwise described herein has a flash point of at least 90° C., e.g., at least 95° C., at least 100° C., or at least 105° C., measured in accordance with ASTM D93. A compound that does not have a flash point below 50° C. is considered to have a flash point above 50° C. for the purposes of this disclosure, even if no flash point is measurable for the material (i.e., due to decomposition at a temperature below where a flash point is reached).

In various embodiments, a compound as otherwise described herein has a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

In various embodiments, a compound as otherwise described herein has a kinematic viscosity at 40° C. in the range of 2 to 20 cSt, e.g., in the range of 2 to 15 cSt, or 3 to 20 cSt, or 3 to 15 cSt, or 5 to 20 cSt, or 5 to 15 cSt. In various embodiments, a compound as otherwise described herein has a kinematic viscosity at 40° C. in the range of 2 to 10 cSt, e.g., 2 to 8 cSt, or 2 to 6 cSt, or 3 to 10 cSt, or 3 to 8 cSt, or 3 to 6 cSt, or 5 to 10 cSt, or 5 to 8 cSt, or 5 to 6 cSt, or 6 to 10 cSt, or 8 to 10 cSt, as measured in accordance with ASTM D455. And in various embodiments, a compound as otherwise described herein has a kinematic viscosity at 40° C. in the range of 2 to 5 cSt, e.g., 2 to 4 cSt, or 2 to 3 cSt, or 3 to 5 cSt, or 3 to 4 cSt, or 4 to 5 cSt, as measured in accordance with ASTM D455.

In various such embodiments, a compound as otherwise described herein has a flash point of at least 50° C., for example, at least 60° C. (e.g., at least 70° C., at least 75° C., at least 80° C., or at least 85° C.), measured in accordance with ASTM D93 and a kinematic viscosity at 40° C. in the range of 2 to 20 cSt, e.g., in the range of 2 to 15 cSt, or 3 to 20 cSt, or 3 to 15 cSt, or 5 to 20 cSt, or 5 to 15 cSt, as measured in accordance with ASTM D455.

In various embodiments, a compound as otherwise described herein has a dynamic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

As described below, the compounds of the disclosure can in certain embodiments be used in evaporative cooling methods, i.e., in which part of the mechanism of cooling is evaporation of the compound. Accordingly, in certain embodiments as otherwise described herein, The identity (and thus the boiling point) of each of the one or more compounds can be selected based on desired operating temperatures of the particular system or process under consideration. Thus, in various embodiments as otherwise described herein, the compound has a boiling point in the range of 30-300° C., e.g., 30-275° C., or 30-250° C., or 30-225° C., or 30-200° C., or 30-175° C., or 30-150° C., or 30-125° C., or 30-100° C. In various embodiments as otherwise described herein, the compound has a boiling point in the range of 50-300° C., e.g., 50-275° C., or 50-250° C., or 50-225° C., or 50-200° C., or 50-175° C., or 50-150° C., or 50-125° C., or 50-100° C. In various embodiments as otherwise described herein, the compound has a boiling point in the range of 75-300° C., e.g., 75-275° C., or 75-250° C., or 75-225° C., or 75-200° C., or 75-175° C., or 75-150° C., or 75-125° C., or 75-100° C. In various embodiments as otherwise described herein, the compound has a boiling point in the range of 100-300° C., e.g., 100-275° C., or 100-250° C., or 100-225° C., or 100-200° C., or 100-175° C., or 100-150° C. In various embodiments as otherwise described herein, the compound has a boiling point in the range of 150-300° C., e.g., 150-275° C., or 150-250° C., or 150-225° C., or 150-200° C.

In certain desirable embodiments, the flash point of the compound is at least 10° C. higher than the boiling point of the compound, e.g., at least 20° C. higher, or at least 50° C. higher.

The present inventors have determined that the compounds described herein can be especially useful as thermal management fluids, e.g., for use as a coolant or heat transfer medium, as described in more detail below. Thus, another aspect of the disclosure provides a thermal management fluid comprising one or more compounds of formula (I) as described herein wherein the one or more compounds is present in a total amount in the range of 1 wt % to 100 wt % based on the total weight of the thermal management fluid. The one or more compounds can be present in the thermal management fluids described herein in a variety of amounts. In various embodiments as otherwise described herein, the one or more compounds is present in a total amount in the range of 1 wt % to 100 wt % (e.g., 5 wt % to 100 wt %, or 10 wt % to 100 wt %, or 20 wt % to 100 wt %) based on the total weight of the thermal management fluid. For example, in various embodiments, the one or more compounds is present in a total amount in the range of 50 wt % to 100 wt %, for example, 75 wt % to 100 wt %, or 85 wt % to 100 wt %, or 90 wt % to 100 wt %, or 95 wt % to 100 wt %, or 98 wt % to 100 wt %. For example, in various embodiments of the thermal management fluid as otherwise described herein, the one or more compounds is present in a total amount in the range of 1 wt % to 99.9 wt % (e.g., 5 wt % to 99.9 wt %, or 10 wt % to 99.9 wt %, or 20 wt % to 99.9 wt %), or 50 wt % to 99.9 wt %, for example, 75 wt % to 99.9 wt %, or 85 wt % to 99.9 wt %, or 90 wt % to 99.9 wt %, or 95 wt % to 99.9 wt %, or 98 wt % to 99.9 wt %, based on the total weight of the thermal management fluid. In various embodiments of the thermal management fluid as otherwise described herein, the one or more compounds is present in a total amount in the range of 1 wt % to 99 wt % (e.g., 5 wt % to 99 wt %, or 10 wt % to 99 wt %, or 20 wt % to 99 wt %), or 50 wt % to 99 wt %, for example, 75 wt % to 99 wt %, or 85 wt % to 99 wt %, or 90 wt % to 99 wt %, or 95 wt % to 99 wt %, based on the total weight of the thermal management fluid. In various embodiments of the thermal management fluid as otherwise described herein, the one or more compounds is present in a total amount in the range of 1 wt % to 95 wt % (e.g., 5 wt % to 95 wt %, or 10 wt % to 95 wt %, or 20 wt % to 95 wt %), or 50 wt % to 95 wt %, for example, 75 wt % to 95 wt %, or 85 wt % to 95 wt %, based on the total weight of the thermal management fluid. In various embodiments of the thermal management fluid as otherwise described herein, the one or more compounds is present in a total amount in the range of 1 wt % to 85 wt % (e.g., 5 wt % to 85 wt %, or 10 wt % to 85 wt %, or 20 wt % to 85 wt %), or 50 wt % to 85 wt %, for example, 65 wt % to 85 wt %, or 75 wt % to 85 wt %, based on the total weight of the thermal management fluid.

The person of ordinary skill in the art will appreciate that various combinations of compounds of the disclosure can be used in thermal management fluids of the disclosure. Accordingly, embodiments of compounds described above can be combined in any number and in any combination in thermal management fluids of the disclosure. When two or more compounds are used in a thermal management fluid, the relative amounts of the two can be varied based on the disclosure herein, depending on the effect desired. In various embodiments, the mass ratio of a first compound to a second compound is in the range of 1:9 to 9:1 (e.g., 1:5 to 5:1, or 1:5 to 1:1, or 1:1 to 5:1).

As the person of ordinary skill in the art will appreciate, the thermal management fluids of the disclosure can also include a variety of other components, such as those conventionally used in compositions for thermal management applications. For example, the thermal management fluid may further include an oil, e.g., a mineral oil, a synthetic oil, or a silicone oil. For example, in various embodiments, the oil is a low-viscosity Group II, III, IV, or V base oil as defined by the American Petroleum Institute (API Publication 1509). These are shown in Table 1.

TABLE 1
Base Oil Stocks API Guidelines

| | | Sulfur | Viscosity |
|---|---|---|---|
| | Saturates | content | Index (VI) |
| Group I | <90 and/or | >300 ppm and | ≥80 and <120 |
| Group II | ≥90 and | ≤300 ppm and | ≥80 and <120 |
| Group III | ≥90 and | ≤300 ppm and | ≥120 |

| | |
|---|---|
| Group IV | Includes polyalphaolefins (PAO) and GTL |
| | (gas-to-liquid) products |
| Group V | All other base oils not included in |
| | Groups I, II, III or IV |

Group II and Group III base oils (such as hydrocracked and hydroprocessed base oils as well as synthetic oils such as hydrocarbon oils, polyalphaolefins, alkyl aromatics, and synthetic esters) and Group IV base oils (such as polyalphaolefins (PAO)) are well known base oils. Oils suitable for use as transformer oils can, in many embodiments, be suitable for use in the compositions, systems and methods of the disclosure. For example, esters also form a useful base oil stock, including synthetic esters, as do GTL (gas-to-liquid) materials, particularly those derived from a hydrocarbon source. For example, the esters of dibasic acids with monoalcohols, or the polyol esters of monocarboxylic acid may be useful as base stocks of the disclosure. Bio-derived oils such as fatty acid methyl esters may also be used.

In various embodiments, the thermal management fluid of the disclosure further comprises a Group II, Group III, Group IV, or Group V base oil. For example, in various embodiments, the thermal management fluid of the disclosure further comprises a Group II or Group III base oil. In various other embodiments, the thermal management fluid of the disclosure further comprises a Group IV base oil such as polyalphaolefins (PAO). In various other embodiments, the thermal management fluid of the disclosure further comprises an ester base oil stock.

The thermal management fluids of the disclosure can be provided as a combination of a combination of the compounds of the disclosure and a base dielectric fluid. Various dielectric fluids known in the art can suitably be used in the compositions, systems and methods described herein. In certain desirable embodiments, the one or more dielectric fluids are non-reactive or otherwise inert with respect to components of a battery such as of a lithium-ion battery.

In certain embodiments as otherwise described herein, the dielectric fluid may be diesel formulated to a high flash point and optionally low sulfur content (e.g., less than 3000 ppm, less than 2000 ppm, or less than 1000 ppm).

In certain embodiments as otherwise described herein, each of the one or more dielectric fluids is an oil, e.g., a mineral oil, a synthetic oil, or a silicone oil. For example, in certain embodiments, the dielectric fluid is a low-viscosity Group III or IV base oil as defined by the American Petroleum Institute (API Publication 1509). Group III base oils (such as hydrocracked and hydroprocessed base oils as well as synthetic oils such as hydrocarbon oils, polyalphaolefins, alkyl aromatics, and synthetic esters) and Group IV base oils (such as polyalphaolefins (PAO)) are wells known base oils.

Oils suitable for use as transformer oils can, in many embodiments, be suitable for use as dielectric fluids in the compositions, systems and methods of the disclosure. Commercially available dielectric fluids include Perfecto™ TR UN (available from Castrol Industrial, United Kingdom) and MIDEL 7131 (available from M&I Materials Ltd., United Kingdom). Examples of commercially available base oils include YUBASE 3 and YUBASE 4 (available from SK Lubricants Co. Ltd., South Korea), DURASYN® 162 and DURASYN® 164 (available from INEOS Oligomers, Houston, Texas), and PRIOLUBE™ oils (available from CRODA, United Kingdom).

The one or more compounds of formula (I) can be homogeneously dispersed within the thermal management fluids of the disclosure. This means that the one or more compounds may be present as small particles (e.g. droplets up to 10 μm, up to 50 μm, or even up to 100 μm in diameter) that are evenly (or homogeneously) mixed throughout the thermal management fluid, or that the one or more compounds is essentially dissolved in the thermal management fluid. It is understood that the one or more compounds can be homogenously dispersed yet leave a minor residue undispersed, but this will be a very small amount, i.e., less than 1%, or 0.5%, or even 0.1% by weight of the compound of formula (I).

Based on the disclosure herein, the one or more dielectric fluids can be selected to provide the thermal management fluids of the disclosure with a desirable overall heat capacity and thermal conductivity. Moreover, the one or more dielectric fluids can be selected to have low reactivity with respect to the other components of the systems in which they are used, and to provide the thermal management fluid with a desired viscosity. Other considerations when selecting the one or more dielectric fluids may include their dielectric constant, toxicity, environmental impact and cost.

In various embodiments, the thermal management fluid of the disclosure further comprises one or more of corrosion inhibitors, anti-oxidants (such as phenolic and aminic anti-oxidants), pour point depressants, antifoams, defoamers, viscosity index modifiers, preservatives, biocides, surfactants, seal swell additives, and combinations thereof. In various embodiments, corrosion inhibitors, anti-oxidants (such as phenolic and aminic anti-oxidants), pour point depressants, antifoams, defoamers, viscosity index modifiers, preservatives, biocides, surfactants, seal swell additives, and combinations thereof, for example, may be present in an amount up to 0.5 wt %, up to 1.0 wt %, or up to 5.0 wt %, based on the total weight of the thermal management fluid. In various such embodiments, one or more of corrosion inhibitors, anti-oxidants (such as phenolic and aminic anti-oxidants), pour point depressants, antifoams, defoamers, viscosity index modifiers, preservatives, biocides, surfactants, seal swell additives, and combinations thereof are present in an amount in the range of 0.2 wt % to 5.0 wt %, e.g., 1.0 wt % to 2.0 wt %, or 0.2 wt % to 1.0 wt %, or 0.2 wt % to 0.5 wt %, or 0.05 wt % to 0.2 wt %, based on the total weight of the thermal management fluid. In various embodiments, the thermal management fluid of the disclosure further comprises one or more flame retardants, e.g., in an amount up to 20 wt %, up to 10 wt %, or up to 5 wt %, based on the total weight of the thermal management fluid. In other embodiments, however, no flame retardant is present.

The thermal management fluids of the disclosure are desirably dielectric fluids. As used herein, a dielectric fluid is a liquid at 25° C. and has a dielectric constant of at least 1.5 at 25° C. Dielectric fluids especially desirable for use herein desirably have relatively high thermal conductivity (e.g., at least 0.05 W/m·K, or at least 0.1 W/m·K, or even at least 0.12 W/m·K at 25° C.) and/or relatively high specific heat capacity (e.g., at least 1 J/g·K, or at least 1.2 J/g·K, or even at least 1.5 J/g·K at 25° C.).

The present inventors have noted that many otherwise desirable compounds for thermal management fluids would meet environmental standards. The fluorinated ether based compounds as described herein are derived from 2,3,3,3-tetrafluoroprop-1-ene, a known environmentally benign fluorinated olefin. Thus, the identified fluorinated ether based compounds contain fluorinated functionality known to be biodegradable. Specifically, 2,3,3,3-tetrafluoroprop-1-ene has a global warming potential (GWP) of 4, no ozone depleting potential, and a very short atmospheric lifetime of approximately 11 days. Accordingly, the present inventors expect the fluorinated ethers as described herein derived from 2,3,3,3-tetrafluoroprop-1-ene to have similar advantageous environmental properties.

The present inventors have also noted that desirable thermal management fluids would meet specific coolant requirements (e.g. viscosity, flashpoint, thermal and electric conductivity). These properties include having a high capacity to carry heat away in a temperature range relevant to operation of a particular electrical device or system (e.g., a lithium-ion battery, data center, or high performance computing center), yet have a sufficiently high dielectric constant to be suitable for use in direct cooling of the device or system. Critically, because there is always a risk that oxygen might enter the overall system, desirable thermal management fluids would advantageously have a high flash point, to reduce the risk of ignition. And to provide more efficient heat transfer during the operation, desirable thermal management fluids would advantageously have low viscosity, allowing for better flowability in a particular electrical device or system.

The present inventors have identified fluorinated ether based compounds for thermal management fluid compositions that provide not only a desirably low viscosity but can in some embodiments also have a high flash point, so they can be easily pumped through a system with low-to-no risk of ignition. Moreover, the present inventors have identified fluorinated ether based compounds with low GWP. Specifically, the present inventors recognized that conventional dielectric fluids (e.g., organic or silicone) typically have good thermal conductivity and specific heat capacity but have undesirably high viscosity while alternative fluorinated ether based fluids have high GWP. Typical low-viscosity dielectric fluids, however, generally have unacceptably low flash points (and other ignition properties) making them unsuitable for use as coolants in systems where there is the potential for temperatures to rise where ignition is a risk. The present inventors have determined that the compounds of the disclosure can provide a thermal management fluid that does not have a low flash point, has advantageously low viscosity, and has low GWP. These properties of the thermal management fluid make them particularly suitable, for example, for direct cooling of electrical devices and systems. Moreover, the identified fluorinated ether based compounds contain fluorinated functionality known to biodegrade. Accordingly, the present inventors expect the fluorinated ethers to have low global warming potentials.

The thermal management fluids and methods of the disclosure can have a number of additional advantages over conventional fluids. Notably, the thermal management fluid of the disclosure can also, in various embodiments, provide one or more of desirably high heat conductivity, low risk of ignition, high dielectric constant, and fast temperature response. The thermal management fluid of the disclosure can in various embodiments also have lower surface tension than conventional low-viscosity dielectric fluids.

Thus, in various embodiments, the thermal management fluid has a flash point of at least 50° C., measured in accordance with ASTM D93 (“Standard Test Methods for Flash Point by Pensky-Martens Closed Cup Tester”), and a dielectric constant of at least 1.5 at 25° C.

Because there is always some risk that oxygen might enter the system, the thermal management fluid of the disclosure advantageously has a high flash point to prevent ignition. As described above, the thermal management fluid of the disclosure can have a flash point of at least 50° C., as measured in accordance with ASTM D93. For example, in various embodiments, the thermal management fluid as otherwise described herein has a flash point of at least 60° C., e.g., at least 70° C., at least 75° C., at least 80° C., or at least 85° C., measured in accordance with ASTM D93. In various embodiments, the thermal management fluid as otherwise described herein has a flash point of at least 90° C., e.g., at least 95° C., at least 100° C., or at least 105° C., measured in accordance with ASTM D93. A material that does not have a flash point below 50° C. is considered to have a flash point above 50° C. for the purposes of this disclosure, even if no flash point is measurable for the material (i.e., due to decomposition at a temperature below where a flash point is reached).

A low viscosity is often desired for a thermal management fluid, to simplify the pumping thereof through a system, especially when relatively narrow passageways are used. The person of ordinary skill in the art will, based on the present disclosure, select components to provide a thermal management fluid with a desired viscosity, e.g., to be conveniently conducted through a system. Accordingly, in various embodiments, the thermal management fluid as otherwise described herein has a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455. In various embodiments, the thermal management fluid as otherwise described herein has a kinematic viscosity at 40° C. in the range of 2 to 20 cSt, e.g., in the range of 2 to 15 cSt, or 3 to 20 cSt, or 3 to 15 cSt, or 5 to 20 cSt, or 5 to 15 cSt. In various embodiments, the thermal management fluid as otherwise described herein has a kinematic viscosity at 40° C. in the range of 2 to 10 cSt, e.g., 2 to 8 cSt, or 2 to 6 cSt, or 3 to 10 cSt, or 3 to 8 cSt, or 3 to 6 cSt, or 5 to 10 cSt, or 5 to 8 cSt, or 5 to 6 cSt, or 6 to 10 cSt, or 8 to 10 cSt, as measured in accordance with ASTM D455. In various embodiments, the thermal management fluid as otherwise described herein has a kinematic viscosity at 40° C. in the range of 2 to 5 cSt, e.g., 2 to 4 cSt, or 2 to 3 cSt, or 3 to 5 cSt, or 3 to 4 cSt, or 4 to 5 cSt, as measured in accordance with ASTM D455. And in various embodiments, the thermal management fluid as otherwise described herein has a dynamic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

The thermal management fluids of the disclosure are desirably dielectric, so that they can be used in direct cooling applications. Accordingly, they have a dielectric constant of at least 1.5 as measured at 25° C. The dielectric constant is measured using the coaxial probe method, using ASTM D924. In various embodiments, the thermal management fluid of the disclosure has a dielectric constant of at least 1.5, e.g., at least 1.75, or at least 2.0, or at least 2.25 as measured at 25° C. In various embodiments, the thermal management fluid of the disclosure has a dielectric constant in the range of 1.5 to 10, or 1.8 to 10, or 1.5 to 2.8, or 1.8 to 2.8.

In various embodiments of the disclosure, the thermal management fluid of the disclosure may have a density of no more than 2.0 g/cm³ at 25° C. For example, in various embodiments of the disclosure, the thermal management fluid as otherwise described herein may have density of no more than 1.8 g/cm³ at 25° C., e.g., no more than 1.6 g/cm³ at 25° C. In various embodiments of the disclosure, the thermal management fluid as otherwise described herein may have a density in the range of about 0.5 to 2.0 g/cm³ at 25° C., e.g., in the range of about 0.6 to 1.9 g/cm³ at 25° C., in the range of about 0.7 to 1.8 g/cm³ at 25° C., in the range of about 0.8 to 1.7 g/cm³ at 25° C., in the range of about 0.9 to 1.6 g/cm³ at 25° C., or in the range of about 0.9 to 1.5 g/cm³ at 25° C.

In various embodiments of the disclosure, the thermal management fluid of the disclosure may have a specific heat capacity of at least 1 J/g·K, or at least 1.2 J/g·K, or even at least 1.5 J/g·K, at 25° C. In various embodiments of the disclosure, the thermal management fluid of the disclosure may have a thermal conductivity in the range of 0.05 W/m·K to 1 W/m·K at 25° C. In various embodiments of the disclosure, the thermal management fluid of the disclosure may have a coefficient of thermal expansion is no more than 1100×10⁻⁶/K (e.g., no more than 1050×10⁻⁶/K, or no more than 1000×10⁻⁶/K).

In various such embodiments of the disclosure, the thermal management fluid has a flash point of at least 50° C., e.g., at least 60° C., at least 65° C., at least 70° C., or at least 75° C., measured in accordance with ASTM D93 and has a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

In various such embodiments of the disclosure, the thermal management fluid has a flash point of at least 50° C., e.g., at least 60° C., at least 65° C., at least 70° C., or at least 75° C., measured in accordance with ASTM D93 and has a kinematic viscosity at 40° C. in the range of 2 to 20 cSt, e.g., in the range of 2 to 15 cSt, or 3 to 20 cSt, or 3 to 15 cSt, or 5 to 20 cSt, as measured in accordance with ASTM D455.

Another aspect of the disclosure provides a method comprising contacting a thermal management fluid as described herein with a surface having a temperature of at least 25° C., e.g. of at least 30° C., or at least 40° C., the surface being in substantial thermal communication with a heat source, and absorbing thermal energy in the thermal management fluid from the heat source through the surface.

The contacting of the thermal management fluid with the surface can be dynamic or static (i.e., conductive). For example, in various embodiments, the contacting of the thermal management fluid with the surface can be performed by circulating, e.g., by pumping or otherwise flowing, the fluid over the surface. In various embodiments, the contacting can also be performed without circulation, e.g., by contacting the surface with the thermal management fluid that is a stationary body of fluid.

The temperature of the surface can vary; the thermal management fluid can be adapted for use with a variety of temperatures. In various embodiments as otherwise described herein, the temperature of the surface is in the range of 25° C. to 150° C., e.g., 25° C. to 100° C., or 25° C. to 90° C., or 25° C. to 85° C., or 25° C. to 80° C., or 25° C. to 75° C., or 25° C. to 70° C. In various embodiments as otherwise described herein, the temperature of the surface is in the range of 30° C. to 150° C., e.g., 30° C. to 100° C., or 30° C. to 90° C., or 30° C. to 85° C., or 30° C. to 80° C., or 30° C. to 75° C., or 30° C. to 70° C. In various embodiments as otherwise described herein, the temperature of the surface is in the range of 40° C. to 150° C., e.g., 50° C. to 150° C., or 60° C. to 150° C., or 70° C. to 150° C., or 80° C. to 150° C., or 90° C. to 150° C., or 100° C. to 150° C., or 110° C. to 150° C. The temperature of the surface in various embodiments (and at various times during operation of a device or system) is no more than a boiling point of any of the one or more compounds of formula (I) of the thermal management system. In various embodiments, throughout the contacting, each of the one or more compounds of formula (I) does not reach its boiling point. However, as described below, in other embodiments, the surface is higher than the boiling point one or more compounds of formula (I), such that evaporation is part of the method of cooling.

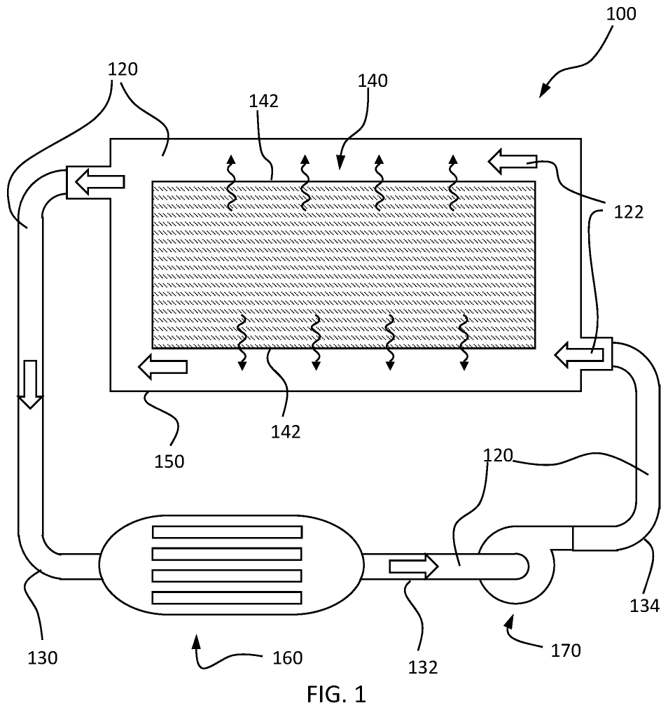

An embodiment of the method of the disclosure is illustrated with reference to FIG. 1. A thermal management circuit 100 is shown in a schematic cross-sectional side view in FIG. 1. The thermal management circuit 100 includes a thermal management fluid 120 that is circulated through the circuit and passes over surface 142. The temperature of surface 142 is elevated in comparison to the temperature of thermal management fluid 120. As a result, thermal energy is absorbed in thermal management fluid 120 from surface 142.

In various embodiments as otherwise described herein, the method includes producing the thermal energy by operating an electrical component. For example, thermal management circuit 100 is associated with electrical component 140, which produces heat during operation. In various embodiments the heat is produced as elements of the electrical component charge and discharge. As will be understood by those of ordinary skill in the art, inefficiencies in the operation of the electrical component and resistances in the circuits corresponding circuits create heat as current passes through the circuits and elements of the electrical component. For example, the heat from the operation of electrical component 140 causes surface 142 to rise in temperature, which then results in the transfer of thermal energy to thermal management fluid 120. In other embodiments, the thermal energy is produced by a chemical reaction, such as an exothermic reaction, or by friction. In still other embodiments, the thermal management fluid is chilled and absorbs thermal energy from surfaces at ambient or slightly elevated temperatures.

In various embodiments as otherwise described herein, the electrical component includes a battery system, a capacitor, inverter, electrical cabling, a fuel cell, a motor, or a computer. For example, in various embodiments the electrical component is a battery system that includes one or more electrochemical cells disposed in a housing. In other embodiments the electrical component is one or more capacitors, such as an electrolytic capacitor or an electric double-layer capacitor, e.g., a supercapacitor. In still other embodiments, the electrical component is one or more fuel cells, such as a polymer electrolyte membrane fuel cell, a direct methanol fuel cell, an alkaline fuel cell, a phosphoric acid fuel cell, a molten carbonate fuel cell, a solid oxide fuel cell, or a reversible fuel cell. In various embodiments the electrical component is an electric motor. In other embodiments, the electrical component is a computer, for example a personal computer or a server. Still, in other embodiments, the electrical component is a high power charging equipment.

The electrical component of the disclosure can operate on direct current (DC) or alternating current (AC). In various embodiments as otherwise described herein, the electrical component operates at DC or AC voltage above 48 V. In various embodiments as otherwise described herein, the electrical component operates at DC or AC voltage above 100 V, above 200 V, or above 300 V.

In various embodiments as otherwise described herein, the surface is a surface of the electrical component. For example, in FIG. 1 a housing of 150 of electrical component 140 contains a reservoir of thermal management fluid 120. Elements of the electrical component including various circuits that produce heat is submerged in thermal management fluid 120 and the thermal management fluid absorbs thermal energy directly from an outside surface 142 of the electrical component 140.

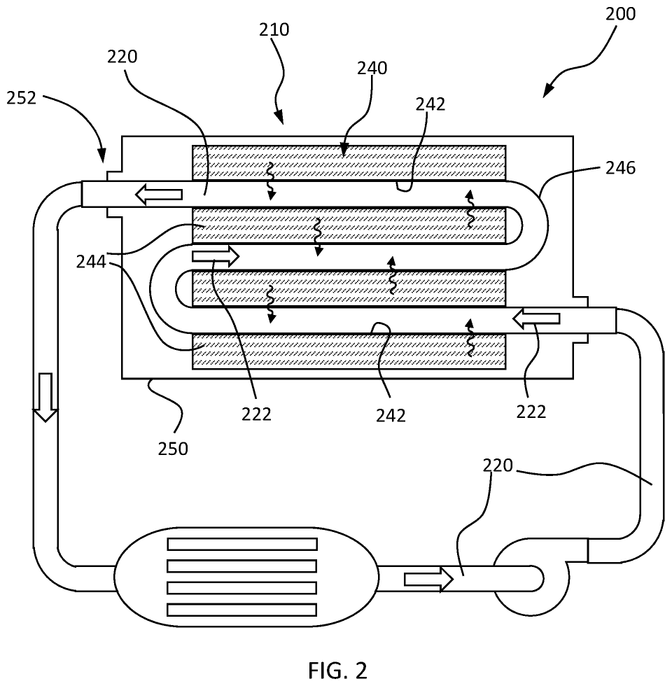

In various embodiments as otherwise described herein, the surface is an internal surface of a conduit. For example, FIG. 2 shows a thermal management circuit 200 that includes electrical component 240 that includes a plurality of individual units 244. In particular, the electrical component 240 is a battery that includes a plurality of electrochemical cells 244. Electrical component 240 further includes a conduit 246 that extends through the inside of the electrical component and between the electrochemical cells 244. As the electrical component produces thermal energy, the internal surface 242 of the conduit 246 is heated and the thermal energy is absorbed by the thermal management fluid 220.

In various embodiments as otherwise described herein, the conduit passes through a housing that surrounds the electrical component. For example, conduit 246 in thermal management circuit 200 extends through apertures 252 in the housing 250 surrounding electrical component 240, which allow thermal management fluid 220 to be conveyed to other elements of the thermal management circuit 200.

Another aspect of the disclosure provides a battery system including: a housing; one or more electrochemical cells disposed in the housing; a fluid path extending through the housing and in substantial thermal communication with the one or more electrochemical cells; and a thermal management fluid according to any of the embodiments described above that is disposed in the fluid path. For example, thermal management circuit 200 in FIG. 2 includes battery system 210. The battery system includes a plurality of electrochemical cells 244 that are disposed inside housing 250. A conduit 246 forms a fluid path that extends through the housing. Thermal management fluid 220 disposed in conduit 246 is thereby placed in thermal communication with the electrochemical cells 244. As the electrochemical cells 244 charge and discharge they produce heat which is absorbed by the thermal management fluid 220. In various embodiments the electrochemical cells are subject to fast charging which yields a large amount of heat. The high heat capacity of the thermal management fluid is able to absorb this large amount of heat as quickly as it is produced.

In various embodiments as otherwise described herein, the fluid path is at least partially defined by a cavity of the housing. For example, in various embodiments at least a portion of the fluid path is formed between the electrochemical cells and the inside wall of the housing, similar to fluid path 122 in component 140.

In various embodiments as otherwise described herein, the fluid path is at least partially defined by at least one conduit disposed in the housing. For example, in battery system 210, conduit 246 provides the fluid path 222 through the housing 250.

In various embodiments as otherwise described herein, the electrochemical cells are lithium-ion electrochemical cells. In other embodiments, the electrochemical cells are solid state cells, lithium-sulfur cells, lithium iron phosphate cells, lithium-ion polymer cells, sodium-ion cells, aluminum-ion cells, lead-acid cells, or magnesium-ion cells.

In various embodiments as otherwise described herein, the battery system is a component of an electric vehicle. In some embodiments, the electric vehicle is a fully electric vehicle or a hybrid electric vehicle. In other embodiments the battery system is a component of a power motor, for example an electric motor or a motor in power electronics. In other embodiments the battery system is part of a stationary energy storage solution, for example a home energy storage solution that operates in cooperation with local renewable energy sources, such as solar panels or wind turbines.

Another aspect of the disclosure provides a thermal management circuit including a fluid path extending around and/or through a heat source; a thermal management fluid of according to any of embodiments described above, disposed in and configured to circulate in the fluid path and to absorb thermal energy produced by the heat source, wherein the fluid is disposed in the fluid path, the heat exchanger, the pump and the connecting duct. For example, thermal management circuit 100 shown in FIG. 1 includes a fluid path 122 that runs around electrical component 140. Thermal management fluid 120 flows through path 122 absorbing thermal energy from electronic component 140. From fluid path 122, the thermal management fluid 120 flows through a first duct 130 to heat exchanger 160. Thermal energy that has accumulated in thermal management fluid 120 is removed from the fluid within heat exchanger 160 before the fluid flows through a second duct 132 to pump 170. After pump 170, the thermal management fluid 120 passes through a third duct 134 returning it to fluid path 122 surrounding electrical component 140. Circuit 100, shown in FIG. 1, is a schematic depiction of an uncomplicated embodiment employing the described thermal management fluid. In other embodiments, the thermal management circuit includes additional elements, such as any combination of valves, pumps, heat exchangers, reservoirs and ducts.

In various embodiments of the as otherwise described herein, the heat source is a battery including a plurality of electrochemical cells, and wherein the fluid path passes between at least two of the electrochemical cells.

In various embodiments as otherwise described herein, the fluid path is defined by a housing around the electrical component. For example, housing 150 in FIG. 1 surrounds electrical component 140 and provides a cavity for thermal management fluid 120. Electrical component 140 is held in the housing at a distance from the walls of housing 150, which allows a path for thermal management fluid 120 to form between the housing 150 and the electrical component 140. While housing 150 has an enclosed shape with specific apertures 152 providing access for thermal management fluid 120, in other embodiments the top of the housing is open and the thermal management fluid is retained in the housing by gravity.

In various embodiments as otherwise described herein, the fluid path is configured to position the thermal management fluid in substantial thermal communication with the electrical component so as to absorb thermal energy produced by the electrical component. For example, in thermal management circuit 100 fluid path 122 extends around electrical component 140 and is in direct contact with the surfaces of electrical component 140. Further, in thermal management circuit 200 fluid path 222 passes through a conduit 246 that runs adjacent to the elements of electrical component 240. In both cases, the fluid path places thermal management fluid in close proximity to the electrical component so that the thermal management fluid readily absorbs thermal energy from the component.

In various embodiments as otherwise described herein, the thermal management circuit further includes a heat exchanger in fluid communication with the fluid path, wherein the thermal management fluid is configured to circulate between the fluid path and the heat exchanger to dissipate heat through the heat exchanger. In various embodiments as otherwise described herein, the heat exchanger is configured to remove heat from the thermal management fluid. For example, in thermal management circuit 100, after thermal management fluid 120 is pumped out of housing 150 it passes to heat exchanger 160 where the thermal energy is transferred to a cooler fluid, such as ambient air or a cooling liquid.

In various embodiments as otherwise described herein, the thermal management circuit includes a battery system according to any of the embodiments described above. For example, thermal management circuit 200 includes battery system 210. In various embodiments as otherwise described herein, the thermal management circuit includes an immobilized desiccant material disposed according to any of the embodiments described above. For example, thermal management circuit 300 includes battery desiccant material 360. In various embodiments, the thermal management circuit is a component of a data center hardware unit. In various embodiments, the thermal management circuit is a component of a high performance computer.

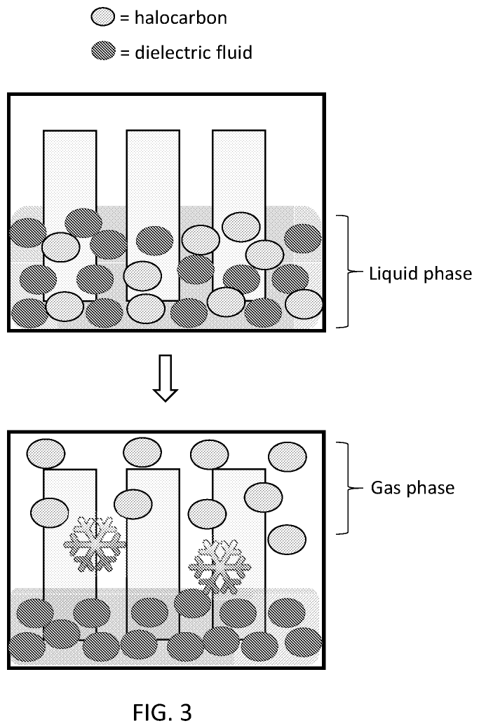

The present inventors have identified dielectric thermal management fluid compositions that utilize the phase change and chemical inertness properties of certain compounds as described herein with the superior dielectric properties and thermal conductivity of organic dielectric fluids. Specifically, the present inventors recognized that certain compounds can undergo a phase change (i.e., liquid to gas) at temperatures relevant to the operation of electrical devices and systems such as lithium-ion batteries. This phase change can be used in a cooling system, with the latent heat of vaporization being used to provide cooling of an electrical component, as schematically shown in FIG. 3. Moreover, many compounds have high flash points, or even no flash point at all. Thus, even though the vaporization of compounds can create a high concentration of compound vapor in the system, there is little risk of ignition of the vapor. Compounds can also generally have advantageously low viscosities and high densities. Many compounds, however, have poor thermal conductivity and specific heat capacity. By comparison, dielectric fluids (e.g., organic or silicone) typically have good thermal conductivity and specific heat capacity. The present inventors have determined that vaporization-based cooling as described herein can be advantageously provided by one or more suitable compounds dispersed in one or more suitable dielectric fluids. In some embodiments, it is the synergistic combination of compound with dielectric fluid that results in the improved thermal management fluid of the disclosure, with the compound component providing vaporization-based cooling without risk of ignition, and the dielectric fluid component providing desirable heat flow and handling properties, and both fluids providing the dielectric properties necessary for direct cooling of electrical devices and systems.

The thermal management fluids and methods of this aspect of the disclosure can have a number of advantages over conventional fluids. Notably, vaporization typically requires much more energy than mere temperature increase of a fluid. Accordingly, because the mechanism of cooling can include the vaporization of the compound component of the dielectric thermal management fluid, the thermal management fluids can have a high overall capacity for cooling. The vaporization of the compound component can also provide a high rate of cooling, which can be especially desirable in the context of lithium-ion batteries to help protect against thermal runaway. And because a compound component can be selected with a desired boiling point, the person of ordinary skill in the art can provide fluids that have high heat capacities at one or more desired temperatures, in order to maintain the temperature of an electrical device or system within a desired operating range. The combination of materials in the dielectric fluids of the disclosure can also, in various embodiments, provide one or more of desirably low viscosity, high heat conductivity, low risk of ignition, high dielectric constant, high density and faster temperature response.

The present inventors have noted that, in certain cases, desirable dielectric thermal management fluids would have a high capacity to carry heat away in a temperature range relevant to operation of a particular electrical device or system (e.g., a lithium-ion battery), yet have a sufficiently high dielectric constant to be suitable for use in direct cooling of the device or system. Moreover, because there is always a risk that oxygen might enter the overall system, desirable thermal management fluids would advantageously have a high or ideally no flash point, to reduce the risk of ignition.

In various such embodiments, one or more of the compounds has a boiling point (i.e. at 1 atm) in the range of 30° C. to 300° C. The inventors have noted that relatively volatile compounds like those described here can provide a cooling effect when they vaporize from liquid to gas (i.e., as measured by their heats of vaporization). This phase transition will occur in a very narrow temperature range, and thus can serve to provide the thermal management fluid with the ability to absorb a relatively large amount of heat at a given temperature (i.e., near the boiling point of the compound, in some embodiments modified by the pressure within the space in which the thermal management fluid is contained). Thus, the use of one or more compounds as provided herein can help to prevent thermal runaway of an electrical component by absorbing a relatively high amount of heat at one or more temperatures. Similarly, the use of one or more compounds as provided herein can help to quickly absorb heat evolved in a fast charging of an electrical component such as a rechargeable battery (e.g., a lithium-ion battery).

Notably, the pressure of the space in which the one or more compounds is contained can be regulated to provide desirable boiling point(s) for the one or more compounds. As the person of ordinary skill in the art will appreciate, the boiling point of a material depends on the pressure, so by regulating the pressure, the boiling point can be modified. The pressure can be regulated, for example, to be greater than atmospheric pressure to reduce the boiling point of a compound. The expansion chambers described herein can be used to regulate pressure in the compound-containing space.

Based on the disclosure herein, the one or more compounds can be selected to have boiling point(s) relevant to the thermal process or system of interest. For example, the each compound can be selected to provide a thermal “stop” to the process or system, helping to maintain temperature around the boiling point thereof even as more heat is absorbed by the thermal management fluid. When multiple compounds are provided, one can provide a thermal “stop” in a desired operation temperature range (e.g., 30-50° C. or 30-80° C., as described above), and another can provide a thermal stop at a higher temperature (e.g., 80-150° C. or 80-110° C., as described above) to prevent thermal runaway. Moreover, the one or more compounds can be selected to have low reactivity with respect to the other components of the systems in which they are used, as well as to provide the overall thermal management fluid with a desired heat capacity, thermal conductivity, and viscosity. Other considerations when selecting the one or more compounds may include toxicity and environmental impact.

Further details regarding evaporative cooling can be found in International Patent Application Publications nos. WO2020007954 and WO2020007955, each of which is hereby incorporated herein by reference in its entirety. In some embodiments, the compounds and thermal management fluids described herein can be used in such methods and apparatuses.

### Preparation of Compounds of Formula (I)

The compounds of the disclosure can be easily prepared from inexpensive starting materials. For example, compounds of formula (I) can be made by contacting a compound of formula (II)

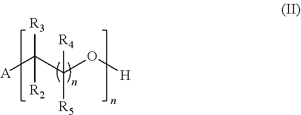

wherein
m is an integer 1, 2, or 3;
n is an integer in the range of 1-16;
A is selected from H, OH, or —O—R₆;
each R₂ and R₃ is independently H or C₁-C₆ alkyl, and each R₄ and R₅ is independently H or C₁-C₆ alkyl, or
each R₂ and R₄ is H or C₁-C₆ alkyl, and R₃ and R₅ together with the carbons to which they are bound come together to form a C₅-C₇ cycloalkyl; and
R₆ is a C₁-C₁₂alkyl;
with CH₂═CF(CF₃) in the presence of a base to provide a first compound of formula (I).

In various embodiments, in one or more of the compounds of formula (II) A is OH. In various embodiments, in one of more of the compounds of formula (II) A is —O—R₆. In various embodiments as otherwise described herein, in one or more compounds of formula (II) R₆ is C₁-C₁₀ alkyl, such as a C₁-C₈ alkyl, C₁-C₆ alkyl, or C₁-C₄ alkyl. In various embodiments as otherwise described herein, in one or more compounds of formula (II) R₆ is C₈-C₁₂ alkyl, such as a C₈-C₁₀ alkyl or C₁₀-C₁₂ alkyl. In various embodiments as otherwise described herein, in one or more compounds of formula (II) R₆ is a branched C₆-C₁₂ alkyl, such as a branched C₆-C₁₀ alkyl, a branched C₆-C₈ alkyl, a branched C₈-C₁₂ alkyl, a branched C₈-C₁₀ alkyl, or a branched C₁₀-C₁₂ alkyl. Branching can be, for example, at an α-position or at a β-position with respect to the oxygen atom to which R₆ is bound. For example, in various embodiments as otherwise described herein, in one or more compounds of formula (II) R₆ is an α-branched C₁-C₁₂ alkyl or is a β-branched C₁-C₁₂ alkyl. In various embodiments, branching is at a β-position with respect to the oxygen atom.

The ethers of formula (I) can generally be made by any convenient route following the general scheme below:

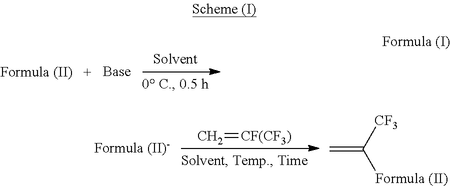

In various embodiments, one or more of the compounds of the present disclosure can be made by further hydrogenating the first compound of formula (I) to provide a second compound of formula (I). Any means of hydrogenating can be use, as known to the skilled person in the art. For example, hydrogenation may be carried out by contacting the first compound of formula (I) with hydrogen gas in the presence of a hydrogenation catalyst to provide a second compound of formula (I) by any convenient route following the general scheme below:

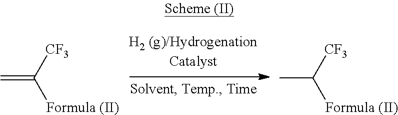

In various embodiments, the hydrogenation catalyst is selected from palladium, platinum, nickel (e.g. Raney nickel) and rhodium catalysts. In various embodiments, the hydrogenation catalyst is selected from palladium and platinum catalysts, such as from Pd/C, Pt(OH)₂, and PtO₂.

In various embodiments, the ethers of formula (I), wherein A is H or —O—R₆ as described herein, can generally be made by any convenient route following scheme (Ill) below:

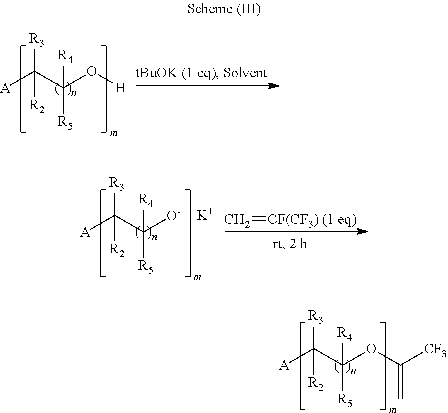

For example, a mono-alcohol of formula (II) (143 mmol) in tetrahydrofuran (150 mL) and dimethylformamide (25 mL) was stirred under nitrogen at room temperature. Potassium tert-butoxide (1 eq) was added portion-wise to the flask. The temperature of the reaction mixture increased from 20° C. to 25° C. during the addition of the base. The flask was then equipped with a solid CO₂/acetone cooled condenser and the reaction mixture was cooled to −60° C. in a solid CO₂/acetone bath. Once cooled, 2,3,3,3-tetrafluoroprop-1-ene (1 eq) gas was added into the solution and the cooling bath was removed. The reaction mixture was allowed to warm up gradually to ambient temperature and stirred for 2 hours. The reaction mixture was then added to an aqueous NaOH solution and the THF was removed under vacuum. The resulting residue was extracted into hexane, dried over sodium sulfate, and the solvent evaporated to produce a red oil. Purification of the oil was accomplished by column chromatograph (silica, 2.5% diethyl ether/hexane) and by distillation to give a colorless oil. The typical reaction conversion for a mono-alcohol of formula (II) to a compound of formula (I) was >95% with a yield of 50-70% after purification.

In various embodiments, the ethers of formula (I), wherein A is OH as described herein, can generally be made by any convenient rout following scheme (IV) below:

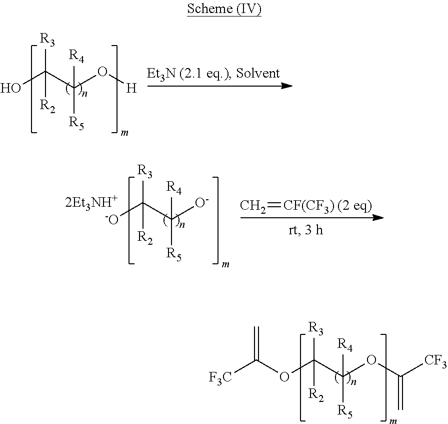

For example, a diol of formula (II) (150 mmol), tetrahydrofuran (165 mL), and triethyl amine (2.1 eq) was stirred under nitrogen at room temperature. The flask was then equipped with a solid CO₂/acetone cooled condenser and the reaction mixture was cooled to −60° C. in a solid CO₂/acetone bath. Once cooled, 2,3,3,3-tetrafluoroprop-1-ene (2 eq) gas was added into the solution and the cooling bath was removed. The reaction mixture was allowed to warm up gradually to ambient temperature and stirred for 3 hours. The reaction mixture was then added to an aqueous NaOH solution and the THF was removed under vacuum. The resulting residue was extracted into hexane and dried over sodium sulfate. The solvent evaporated under vacuum to produce an oil. Purification of the oil was accomplished by column chromatograph (silica, 1% diethyl ether/hexane) and by distillation to give a colorless oil. The typical reaction conversion for a mono-alcohol of formula (II) to a compound of formula (I) was >95% with a yield of 40-75% after purification.

In various embodiments, a one or more of the compounds of the present disclosure can be made by scheme (Ill) or scheme (IV) to provide a first compound of formula (I) can be further hydrogenated as described by scheme (II) to provide a second compound of formula (I). For example, a trifluoromethyl vinyl ether of formula (I) (147 mmol) and 10% Pd/C (0.005 eq) in ethanol (100 mL) and hexane (100 mL) was placed in an autoclave and pressurized to 15 bar hydrogen. The mixture was stirred at 25° C. for 16 hours. The pressure was then released and the mixture was filtered through Celite. The solvent was then evaporated to produce an oil. Purification of the oil was accomplished by distillation to afford the a second compound of formula (I) in >93% yield and a >99% purity, as determined by gas chromatography.

The person of ordinary skill in the art will recognize that a variety of bases can be used in effecting these transformations. While tBuOK and triethyl amine are a desirable bases, others can be used, including NaH, n-BuLi, t-BuLi, and MeMgBr. The person of ordinary skill in the art will also recognize that a variety of solvents can be used in effecting these transformations. While dimethylformamide (DMF) and tetrahydrofuran (THF) is preferable, other solvents can be used. In various embodiments, a one or more of the compound of the present disclosures made by the methods as described herein are prepared under inert atmosphere, for example under an argon atmosphere or a nitrogen atmosphere. The desired compound of formula (I) can be isolated by any known method. Any desired purification can be accomplished by any known method, e.g. distillation.

### Example

The methods of the disclosure are illustrated further by the following example, which is not to be construed as limiting the disclosure in scope or spirit to the specific procedures and compounds described in them.

Compounds of the disclosure include those of Table 1 below:

TABLE 1

| | |
|---|---|
| 1 | 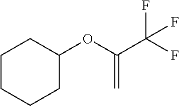 |
| | C₉H₁₃OF₃ |
| 2 | 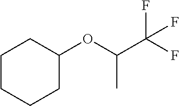 |
| | C₉H₁₅OF₃ |
| 3 | 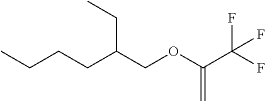 |
| | C₁₁H₁₉OF₃ |
| 4 | 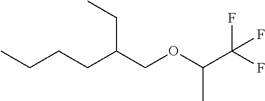 |
| | C₁₁H₂₁OF₃ |
| 5 | 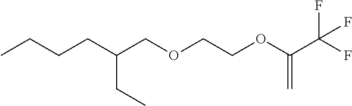 [SMILES: C=C(OCCOCC(CC)CCCC)C(F)(F)F] |
| | C₁₃H₂₃O₂F₃ |
| 6 | 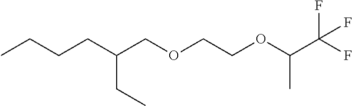 [SMILES: CCCCC(CC)COCCOC(C)C(F)(F)F] |
| | C₁₃H₂₅O₂F₃ |
| 7 | 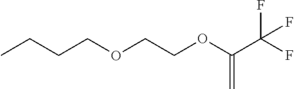 |
| | C₉H₁₅O₂F₃ |
| 8 | 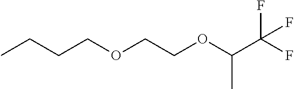 |
| | C₉H₁₇O₂F₃ |
| 9 | 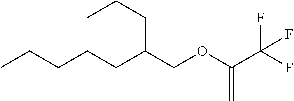 [SMILES: C=C(OCC(CCC)CCCCC)C(F)(F)F] |
| | C₁₃H₂₃OF₃ |
| 10 | 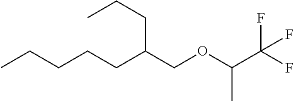 [SMILES: CCCCCC(CCC)COC(C)C(F)(F)F] |
| | C₁₃H₂₅OF₃ |
| 11 | 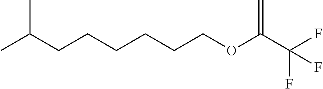 [SMILES: C=C(OCCCCCCC(C)C)C(F)(F)F] |
| | C₁₂H₂₁OF₃ |
| 12 | 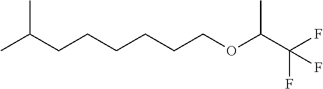 [SMILES: CC(C)CCCCCCOC(C)C(F)(F)F] |
| | C₁₂H₂₃OF₃ |
| 13 | 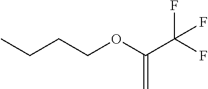 |
| | C₇H₁₁OF₃ |
| 14 | 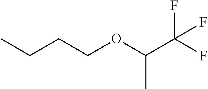 |
| | C₇H₁₃OF₃ |
| 15 | 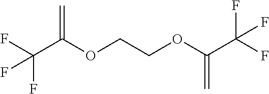 [SMILES: C=C(OCCOC(=C)C(F)(F)F)C(F)(F)F] |
| | C₈H₈O₂F₆ |
| 16 | 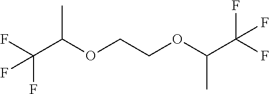 [SMILES: CC(OCCOC(C)C(F)(F)F)C(F)(F)F] |
| | C₈H₁₂O₂F₆ |
| 17 | 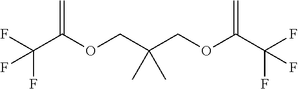 [SMILES: C=C(OCC(C)(C)COC(=C)C(F)(F)F)C(F)(F)F] |
| | C₁₁H₁₄O₂F₆ |
| 18 | 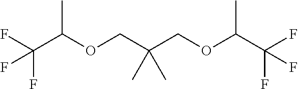 [SMILES: CC(OCC(C)(C)COC(C)C(F)(F)F)C(F)(F)F] |
| | C₁₁H₁₈O₂F₆ |
| 19 | 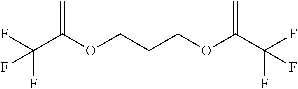 [SMILES: C=C(OCCCOC(=C)C(F)(F)F)C(F)(F)F] |
| | C₉H₁₀O₂F₆ |
| 20 | 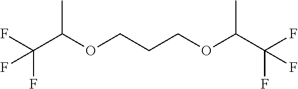 [SMILES: CC(OCCCOC(C)C(F)(F)F)C(F)(F)F] |
| | C₉H₁₄O₂F₆ |
| 21 | 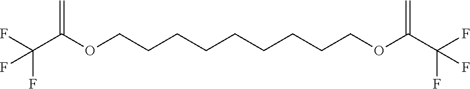 [SMILES: C=C(OCCCCCCCCCOC(=C)C(F)(F)F)C(F)(F)F] |
| | C₁₅H₂₂O₂F₆ |
| 22 | 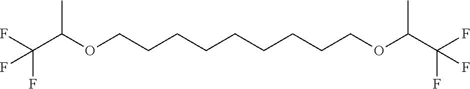 [SMILES: CC(OCCCCCCCCCOC(C)C(F)(F)F)C(F)(F)F] |
| | C₁₅H₂₆O₂F₆ |

Table 2 presents estimations of properties (boiling point, pour point, density at 25° C., dynamic viscosity at 25° C., kinematic viscosities at 25° C., and thermal conductivity at 25° C. mW/m-K for the structures above, based on comparison with the properties of known molecules.

TABLE 2

| | | | | Flash | | | | Thermal |
|---|---|---|---|---|---|---|---|---|
| | Mol Wt | BP | PP | Point | Density | DV25 | KV25 | Cond. |
| # | g/mol | ° C. | ° C. | ° C. | g/cm³ | cP | cSt | mW/mK |
| 1 | 194.20 | 165 | −64 | 60 | 1.256 | 1.503 | 1.197 | 132.333 |
| 2 | 196.21 | 168 | −63 | 29 | 1.248 | 1.540 | 1.234 | 121.086 |
| 3 | 224.27 | 191 | −64 | | 1.036 | 1.679 | 1.621 | 137.201 |
| 4 | 226.28 | 194 | −63 | | 0.931 | 1.744 | 1.873 | 126.724 |
| 5 | 268.32 | 259 | −19 | 111 | 0.945 | 3.143 | 3.326 | 145.161 |
| 6 | 270.34 | 262 | −18 | 86 | 0.964 | 3.323 | 3.447 | 134.328 |
| 7 | 212.21 | 168 | −49 | 67 | 1.048 | 1.110 | 1.059 | 141.130 |
| 8 | 214.23 | 171 | −48 | 38 | 1.039 | 1.056 | 1.016 | 135.556 |
| 9 | 252.32 | 237 | −41 | | 0.925 | 2.172 | 2.348 | 139.198 |
| 10 | 254.34 | 240 | −40 | | 0.923 | 2.423 | 2.625 | 128.854 |
| 11 | 238.29 | 214 | −52 | | 0.923 | 2.637 | 2.857 | 138.526 |
| 12 | 240.31 | 217 | −52 | | 0.929 | 2.600 | 2.799 | 128.215 |

| | | | | Flash | | | | Thermal |
|---|---|---|---|---|---|---|---|---|
| # | Mol Wt | BP | PP | Point | Density | DV25 | KV25 | Cond. |
| 13 | 168.16 | 100 | −94 | | 1.172 | 0.591 | 0.504 | 131.750 |
| 14 | 170.18 | 103 | −93 | | 1.204 | 0.743 | 0.617 | 128.137 |
| 15 | 250.14 | 136 | −72 | 66 | 1.393 | 0.822 | 0.590 | 124.308 |
| 16 | 254.17 | 142 | −70 | 3 | 1.392 | 1.114 | 0.800 | 106.194 |
| 17 | 292.22 | 202 | −36 | | 1.448 | 1.513 | 1.045 | 125.152 |
| 18 | 296.25 | 208 | −34 | | 1.331 | 1.969 | 1.479 | 110.222 |
| 19 | 264.17 | 159 | −61 | | 1.378 | 1.121 | 0.813 | 127.458 |
| 20 | 268.20 | 165 | −59 | | 1.377 | 1.513 | 1.099 | 100.968 |
| 21 | 348.33 | 297 | −60 | | 1.300 | 6.888 | 5.298 | 144.809 |
| 22 | 352.36 | 165 | −64 | | 1.256 | 1.503 | 1.197 | 132.333 |

Table 3 provides a comparison of the flash point between the fluorinated ethers of the present disclosure and comparative non-fluorinated ether analogues. The comparative non-fluorinated ether analogues are designated with a “C” in the table below.

TABLE 3

| | | Flash Point |
|---|---|---|
| # | Structure | (° C.) |
| 1C | 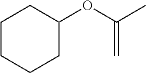 | 55 |
| 1 | 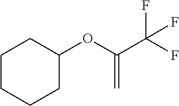 | 60 |
| 2C | 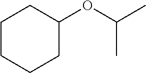 | 38 |
| 2 | 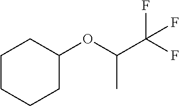 | 29 |
| 5C | 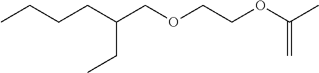 | 108 |
| 5 | 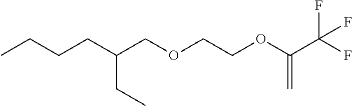 [SMILES: C=C(OCCOCC(CC)CCCC)C(F)(F)F] | 111 |
| 6C | 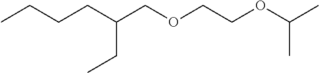 | 93 |
| 6 | 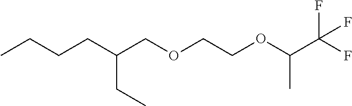 [SMILES: CCCCC(CC)COCCOC(C)C(F)(F)F] | 86 |
| 7C | 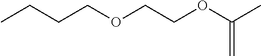 | 63 |
| 7 | 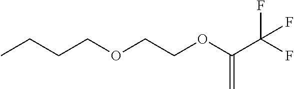 | 67 |
| 8C | 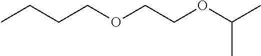 | 47 |
| 8 | 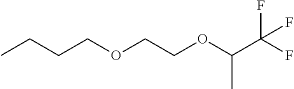 | 38 |
| 15C | 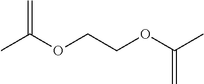 | 57 |
| 15 | 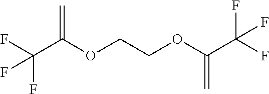 [SMILES: C=C(OCCOC(=C)C(F)(F)F)C(F)(F)F] | 66 |
| 16C | 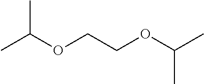 | 23 |
| 16 | 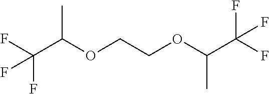 [SMILES: CC(OCCOC(C)C(F)(F)F)C(F)(F)F] | 3 |

From the table above, the general trend can be seen that the unsaturated fluorinated ether compounds have a higher flash point than the corresponding unsaturated and saturated non-fluorinated ether compounds. It is also notable that the saturated fluorinated ether compounds have an unexpectedly lower flashpoint than the saturated non-fluorinated ether compounds. Without being bound by theory, it is hypothesized that the unexpectedly lower flashpoint may be attributed to the electron withdrawing effect the CF₃ group has on the tertiary carbon.

Other aspects of the disclosure are described with respect to the following enumerated embodiments, which may be combined in any fashion and in any number that is not technically or logically inconsistent.

Embodiment 1 is directed to a compound of formula (I):

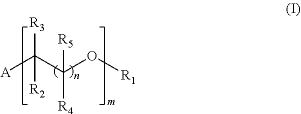

wherein
m is an integer 1, 2, or 3;
n is an integer in the range of 1-16;
A is H or —O—R₆;
R₁ is —C(═CH₂)—CF₃, or —CH(CH₃)—CF₃;
each R₂ and R₃ is independently H or C₁-C₆ alkyl, and each R₄ and R₅ is independently H or C₁-C₆ alkyl, or
each of R₂ and R₄ is H or C₁-C₆ alkyl, and R₃ and R₅ together with the carbons to which they are bound come together to form a C₅-C₇ cycloalkyl; and
R₆ is C₁-C₁₂ alkyl, —C(═CH₂)—CF₃, or —CH(—CH₃)—CF₃.

Embodiment 2 is directed to the compound of embodiment 1, wherein R₁ is —C(═CH₂)—CF₃ and A is —O—R₆.

Embodiment 3 is directed to the compound of embodiment 1, wherein R₁ is —CH(CH₃)—CF₃ and A is —O—R₆.

Embodiment 4 is directed to the compound of any of embodiments 1-3, wherein R₆ is —C(═CH₂)—CF₃.

Embodiment 5 is directed to the compound of any of embodiments 1-3, wherein R₆ is —CH(CH₃)—CF₃.

Embodiment 6 is directed to the compound of embodiment 1, wherein R₁ is —C(═CH₂)—CF₃, A is —O—R₆, and R₆ is —C(═CH₂)—CF₃.

Embodiment 7 is directed to the compound of embodiment 1, wherein R₁ is —CH(CH₃)—CF₃, A is —O—R₆, and R₆ is —CH(CH₃)—CF₃.

Embodiment 8 is directed to the compound of embodiment 1, wherein R₁ is —C(═CH₂)—CF₃, A is —O—R₆, and R₆ is —CH(CH₃)—CF₃.

Embodiment 9 is directed to the compound of embodiment 1, wherein R₁ is —CH(CH₃)—CF₃, A is —O—R₆, and R₆ is —C(═CH₂)—CF₃.

Embodiment 10 is directed to the compound of any of embodiments 1-3, wherein R₆ is C₁-C₁₂ alkyl.

Embodiment 11 is directed to the compound of any of embodiments 1-3, wherein R₆ is C₁-C₁₀ alkyl, such as C₁-C₈ alkyl, C₁-C₆ alkyl, or C₁-C₄ alkyl.

Embodiment 12 is directed to the compound of any of embodiments 1-3, wherein R₆ is C₈-C₁₂ alkyl, such as C₈-C₁₀ alkyl or C₁₀-C₁₂ alkyl.

Embodiment 13 is directed to the compound of any of embodiments 1-3, wherein R₆ is a branched C₆-C₁₂ alkyl, such as a branched C₆-C₁₀ alkyl, a branched C₆-C₈ alkyl, a branched C₈-C₁₂ alkyl, a branched C₈-C₁₀ alkyl, or a branched C₁₀-C₁₂ alkyl.

Embodiment 14 is directed to the compound of embodiment 13, wherein branching of R₆ is at a β-position to the oxygen atom to which R₆ is bound.

Embodiment 15 is directed to the compound embodiment 1, wherein R₁ is —C(═CH₂)—CF₃ and A is H.

Embodiment 16 is directed to the compound of embodiment 1, wherein R₁ is —CH(CH₃)—CF₃ and A is H.

Embodiment 17 is directed to the compound of any of embodiments 1-16, wherein each R₂, R₃, R₄ and R₅ is independently selected from H and C₁-C₄ alkyl (such as C₁-C₃ alkyl or C₁-C₂ alkyl).

Embodiment 18 is directed to the compound of any of embodiments 1-16, wherein each of R₂, R₃, R₄ and R₅ is independently selected from H and C₁ alkyl (i.e., methyl).

Embodiment 19 is directed to the compound of any of embodiments 1-16, wherein each R₂ is H and each R₃ is H or C₁-C₆ alkyl (such as C₁-C₄ alkyl or C₁-C₂ alkyl); and each R₄ is H and each R₅ is H or C₁-C₆ alkyl (such as C₁-C₄ alkyl or C₁-C₂ alkyl).

Embodiment 20 is directed to the compound of any of embodiments 1-16, wherein each R₂ is H, R₃ is H, R₄ is H and each R₅ is H or C₁-C₆ alkyl (such as C₁-C₄ alkyl or C₁-C₂ alkyl).

Embodiment 21 is directed to the compound of any of embodiments 1-16, wherein each R₂ is H, R₄ is H, R₅ is H and R₃ is H or C₁-C₆ alkyl (such as C₁-C₄ alkyl or C₁-C₂ alkyl).

Embodiment 22 is directed to the compound of any of embodiments 1-19, wherein only one R₅ is other than H.

Embodiment 23 is directed to the compound of any of embodiments 1-16, wherein each R₂ and R₄ is H or C₁-C₄ alkyl (such as C₁-C₃ alkyl or C₁-C₂ alkyl), and R₃ and R₅ together with the carbons to which they are bound come together to form a C₅-C₇ cycloalkyl.

Embodiment 24 is directed to the compound of any or embodiments 1-16, wherein each R₂ and R₄ is H or methyl, and R₃ and R₅ together with the carbons to which they are bound come together to form a C₅-C₇ cycloalkyl.

Embodiment 25 is directed to the compound of any of embodiments 1-16, wherein each R₂ and R₄ is H or C, (i.e. methyl), and R₃ and R₅ together with the carbons to which they are bound come together to form a C cycloalkyl.

Embodiment 26 is directed to the compound of any of embodiments 1-16, wherein each of R₂, R₃, R₄, and R₅ is H, i.e., the compound has the formula

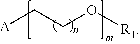

Embodiment 27 is directed to the compound of any of embodiments 1-16, wherein each of R₂, R₄, and R₅ is H, and wherein R₃ is H or C₁-C₆ alkyl (such as methyl or ethyl), i.e., the compound has the formula

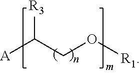

Embodiment 28 is directed to a compound of formula (I):

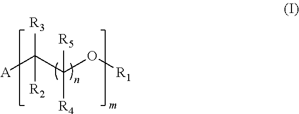

wherein
m is an integer 1, 2, or 3;
n is an integer in the range of 1-16;
A is H or —O—R₆;
R₁ is C(═CH₂)—CF₃ or —CH(CH₃)—CF₃;
each of R₂ and R₃ is independently H or C₁-C₆ alkyl;
each of R₄ and R₅ is independently H or C₁-C₆ alkyl; and
R₆ is C₁-C₁₂ alkyl, —C(═CH₂)—CF₃, or —CH(CH₃)—CF₃
   wherein the various groups can be defined as in any of embodiments 1-22, 26 and 27 above.

Embodiment 29 is directed to a compound of formula (I):

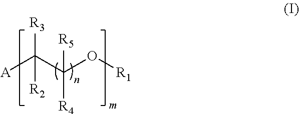

wherein
m is an integer 1, 2, or 3;
n is an integer in the range of 1-16;
A is H or —O—R₆;
R₁ is C(═CH₂)—CF₃, or —CH(CH₃)—CF₃;
each of R₂ and R₄ is H or C₁-C₆ alkyl, and R₃ and R₅ together with the carbons to which they are bound come together to form a C₅-C₇ cycloalkyl; and
R₆ is C₁-C₁₂ alkyl, —C(═CH₂)—CF₃, or —CH(CH₃)—CF₃;
   wherein the various groups can be defined as in any of embodiments 1-16 and 23-25 above.

Embodiment 30 is directed to the compound of any of embodiments 1-29, wherein n is an integer 1-12, e.g., n is an integer 1-8.

Embodiment 31 is directed to the compound of any of embodiments 1-29, wherein n is an integer 1-6, e.g., n is an integer 1-4 or an integer 1-2.

Embodiment 32 is directed to the compound of embodiments 1-29, wherein n is an integer 4-16, e.g., n is an integer 8-16 or an integer 10-16.

Embodiment 33 is directed to the compound of embodiments 1-29, wherein n is an integer 4-12, e.g., n is an integer 6-12.

Embodiment 34 is directed to the compound of any of embodiments 1-29, wherein n is 1, e.g. the compound has the formula:

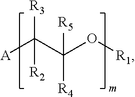

such as

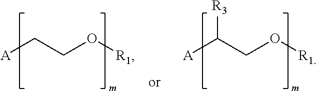

Embodiment 35 is directed to the compound of embodiment 34, wherein A is —O—R₆, i.e., the compound has the formula

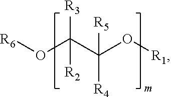

such as

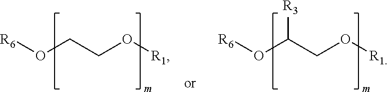

Embodiment 36 is directed to the compound of any of embodiments 1-29, wherein n is 2, i.e., the compounds have the formula

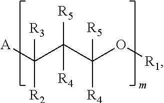

such as

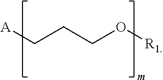

Embodiment 37 is directed to the compound of embodiment 36, wherein A is —O—R₆, each R₂ is H and each R₃ is H, and each R₄ and R₅ is independently H or C₁-C₆ alkyl, i.e., the compound has the formula

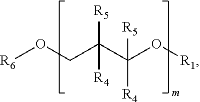

such as

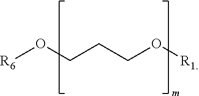

Embodiment 38 is directed to the compound of any of embodiments 1-37, wherein m is 1.

Embodiment 39 is directed to the compound of any of embodiments 1-37, wherein m is 2.

Embodiment 40 is directed to the compound of any of embodiments 1-37, wherein m is 3.

Embodiment 41 is directed to the compound of embodiments 1-14 and 30-37, wherein the compound has the formula:

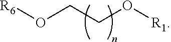

Embodiment 42 is directed to the compound of embodiment 41, wherein n is an integer in the range of 1-16, e.g. in the range of 1-12, in the range of 1-8, in the range of 1-4, on in the range of 1-2.

Embodiment 43 is directed to the compound of embodiment 41, wherein n is 1.

Embodiment 44 is directed to the compound of embodiment 41 or embodiment 42, wherein R₁ is —C(═CH₂)—CF₃ or —CH(CH₃)—CF₃, and R₆ is C₁-C₁₂ alkyl, —C(═CH₂)—CF₃, or —CH(CH₃)—CF₃.

Embodiment 45 is directed to the compound of embodiment 41 or embodiment 42, wherein R₁ is —C(═CH₂)—CF₃.

Embodiment 46 is directed to the compound of embodiment 41 or embodiment 42, wherein R₁ is —CH(CH₃)—CF₃.

Embodiment 47 is directed to the compound of any of embodiments 41-46, wherein R₆ is methyl or ethyl.

Embodiment 48 is directed to the compound of any of embodiments 41-46, wherein R₆ is propyl (e.g., n-propyl, isopropyl) or butyl, e.g., n-butyl, t-butyl, sec-butyl or isobutyl.

Embodiment 49 is directed to the compound of any of embodiments 41-46, wherein R₆ is branched pentyl, e.g., 1,1-dimethylpropyl, 2,2-dimethylpropyl.

Embodiment 50 is directed to the compound of any of embodiments 41-46, wherein R₆ is branched, e.g., a group —CH₂—CH(R_{b})(R_{d}) in which R_{b} is methyl or ethyl, and R_{d} is C₃-C₈ alkyl, e.g., R₆ is 2-ethylhexyl.

Embodiment 51 is directed to the compound of any of embodiments 41-43, wherein R₆ is —C(═CH₂)—CF₃ and R₁ is —C(═CH₂)—CF₃.

Embodiment 52 is directed to the compound of any of embodiments 41-43, wherein R₆ is —CH(CH₃)—CF₃ and R₁ is —CH(CH₃)—CF₃.

Embodiment 53 is directed to the compound of any of embodiments 1-52, wherein the compound contains a total number of carbon atoms from 5 to 30 (e.g., from 5 to 26, from 5 to 22, from 5 to 18, from 6 to 30, from 6 to 26, from 6 to 22, from 8 to 30, from 8 to 26, or from 8 to 22).

Embodiment 54 is directed to the compound of any of embodiments 1-52, wherein the compound contains a total number of carbon atoms from 6-16.

Embodiment 55 is directed to the compound of embodiment 1, wherein the compound is selected from:

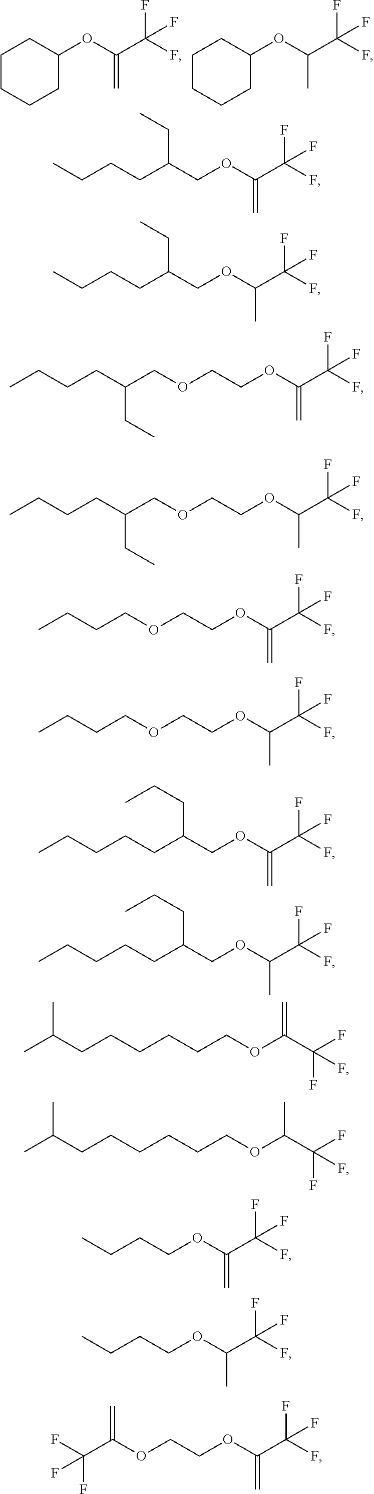

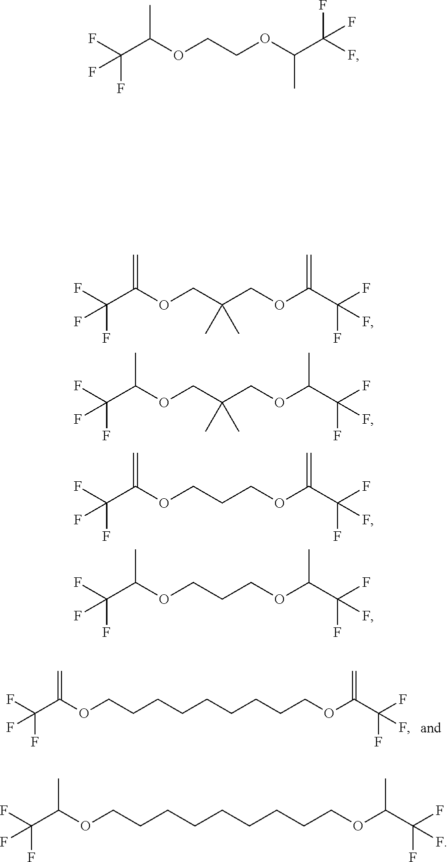

Embodiment 56 is directed to the compound of any of embodiments 1-55, wherein the compound has a flash point of at least 50° C., for example, at least 60° C. (e.g., at least 70° C., at least 75° C., at least 80° C., or at least 85° C.), measured in accordance with ASTM D93.

Embodiment 57 is directed to the compound of any of embodiments 1-55, wherein the compound has a flash point of at least 90° C. (e.g., at least 95° C., at least 100° C., or at least 105° C.), measured in accordance with ASTM D93.

Embodiment 58 is directed to the compound of any of embodiments 1-57, wherein the compound has a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

Embodiment 59 is directed to the compound of any of embodiments 1-57, wherein the compound has a kinematic viscosity at 40° C. in the range of 2 to 20 cSt, e.g., in the range of 2 to 15 cSt, or 3 to 20 cSt, or 3 to 15 cSt, or 5 to 20 cSt, or 5 to 15 cSt, as measured in accordance with ASTM D455.

Embodiment 60 is directed to the compound of any of embodiments 1-57, wherein the compound has a kinematic viscosity at 40° C. in the range of 2 to 10 cSt, e.g., 2 to 8 cSt, or 2 to 6 cSt, or 3 to 10 cSt, or 3 to 8 cSt, or 3 to 6 cSt, or 5 to 10 cSt, or 5 to 8 cSt, or 5 to 6 cSt, or 6 to 10 cSt, or 8 to 10 cSt, as measured in accordance with ASTM D455.

Embodiment 61 is directed to the compound of any of embodiments 1-57, wherein the compound has a kinematic viscosity at 40° C. in the range of 2 to 5 cSt, or 2 to 4 cSt, or 2 to 3 cSt, or 3 to 5 cSt, or 3 to 4 cSt, or 4 to 5 cSt, as measured in accordance with ASTM D455.

Embodiment 62 is directed to the compound of any of embodiments 1-61, wherein the compound has a dynamic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

Embodiment 63 is directed to the compound of any of embodiments 1-55, wherein the compound has a flash point of at least 50° C., for example, at least 60° C. (e.g., at least 70° C., at least 75° C., at least 80° C., or at least 85° C.), measured in accordance with ASTM D93 and a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

Embodiment 64 is directed to the compound of any of embodiments 1-55, wherein the compound has a flash point of at least 50° C., for example, at least 60° C. (e.g., at least 70° C., at least 75° C., at least 80° C., or at least 85° C.), measured in accordance with ASTM D93 and a kinematic viscosity at 40° C. in the range of 2 to 20 cSt, e.g., in the range of 2 to 15 cSt, or 3 to 20 cSt, or 3 to 15 cSt, or 5 to 20 cSt, or 5 to 15 cSt, as measured in accordance with ASTM D455.

Embodiment 65 is directed to the compound of any of embodiments 1-64, wherein the compound has a boiling point in the range of 30-300° C., e.g., 30-275° C., or 30-250° C., or 30-225° C., or 30-200° C., or 30-175° C., or 30-150° C., or 30-125° C., or 30-100° C.

Embodiment 66 is directed to the compound of any of embodiments 1-64, wherein the compound has a boiling point in the range of 50-300° C., e.g., 50-275° C., or 50-250° C., or 50-225° C., or 50-200° C., or 50-175° C., or 50-150° C., or 50-125° C., or 50-100° C.

Embodiment 67 is directed to the compound of any of embodiments 1-64, wherein the compound has a boiling point in the range of 75-300° C., e.g., 75-275° C., or 75-250° C., or 75-225° C., or 75-200° C., or 75-175° C., or 75-150° C., or 75-125° C., or 75-100° C.

Embodiment 68 is directed to the compound of any of embodiments 1-64, wherein the compound has a boiling point in the range of 100-300° C., e.g., 100-275° C., or 100-250° C., or 100-225° C., or 100-200° C., or 100-175° C., or 100-150° C.; or in the range of 150-300° C., e.g., 150-275° C., or 150-250° C., or 150-225° C., or 150-200° C.

Embodiment 69 is directed the compound of any of embodiments 1-68, wherein the flash point of the compound is at least 10° C. higher than the boiling point of the compound, e.g., at least 20° C. higher, or at least 50° C. higher.

Embodiment 70 is directed to a thermal management fluid comprising one or more compounds of any of embodiments 1-98, the one or more compounds being present in a total amount in the range of 1 wt % to 100 wt %, based on the total weight of the thermal management fluid.

Embodiment 66 is directed to the thermal management fluid of embodiment 65, wherein the one or more compounds is present in an amount in the range of 5 wt % to 100 wt %, or 10 wt % to 100 wt %, or 20 wt % to 100 wt %, based on the total weight of the thermal management fluid.

Embodiment 67 is directed to the thermal management fluid of embodiment 65, wherein the one or more compounds is present in an amount in the range of 50 wt % to 100 wt %, for example, 75 wt % to 100 wt %, or 85 wt % to 100 wt %, or 90 wt % to 100 wt %, or 95 wt % to 100 wt %, or 98 wt % to 100 wt %, based on the total weight of the thermal management fluid.

Embodiment 68 is directed to the thermal management fluid of embodiment 65, wherein the one or more compounds is present in an amount in the range of 1 wt % to 99.9 wt % (e.g., 5 wt % to 99.9 wt %, or 10 wt % to 99.9 wt %, or 20 wt % to 99.9 wt %), or 50 wt % to 99.9 wt %, for example, 75 wt % to 99.9 wt %, or 85 wt % to 99.9 wt %, or 90 wt % to 99.9 wt %, or 95 wt % to 99.9 wt %, or 98 wt % to 99.9 wt %, based on the total weight of the thermal management fluid.

Embodiment 69 is directed to the thermal management fluid of embodiment 65, wherein the one or more compounds is present in an amount in the range of 1 wt % to 99 wt % (e.g., 5 wt % to 99 wt %, or 10 wt % to 99 wt %, or 20 wt % to 99 wt %), or 50 wt % to 99 wt %, for example, 80 wt % to 99 wt %, or 85 wt % to 99 wt %, or 90 wt % to 99 wt %, or 95 wt % to 99 wt %, based on the total weight of the thermal management fluid.

Embodiment 70 is directed to the thermal management fluid of embodiment 65, wherein the one or more compounds is present in an amount in the range of 1 wt % to 95 wt % (e.g., 5 wt % to 95 wt %, or 10 wt % to 95 wt %, or 20 wt % to 95 wt %), or 50 wt % to 95 wt %, for example, 75 wt % to 95 wt %, or 85 wt % to 95 wt %, based on the total weight of the thermal management fluid.

Embodiment 71 is directed to the thermal management fluid of embodiment 65, wherein the one or more compounds is present in an amount in the range of 1 wt % to 85 wt % (e.g., 5 wt % to 85 wt %, or 10 wt % to 85 wt %, or 20 wt % to 85 wt %), or 50 wt % to 85 wt %, for example, 65 wt % to 85 wt %, or 75 wt % to 85 wt %, based on the total weight of the thermal management fluid.

Embodiment 72 is directed to the thermal management fluid of any of embodiments 65-71, further comprising a Group II, Group III, Group IV, or a Group V base oil.

Embodiment 73 is directed to the thermal management fluid of any of embodiments 65-71, further comprising a Group II or Group III base oil.

Embodiment 74 is directed to the thermal management fluid of any of embodiments 65-71, further comprising a Group IV base oil (such as polyalphaolefins (PAO)).

Embodiment 75 is directed to the thermal management fluid of any of embodiments 65-74, further comprising an ester base oil stock.

Embodiment 76 is directed to the thermal management fluid of any of embodiments 65-75, further comprising one or more of corrosion inhibitors, anti-oxidants (such as phenolic and 55 anti-oxidants), pour point depressants, antifoams, defoamers, viscosity index modifiers, preservatives, biocides, surfactants, seal swell additives, and combinations thereof, e.g., in an amount up to 0.5 wt %, up to 1.0 wt %, or up to 5.0 wt %.

Embodiment 77 is directed to the thermal management fluid of any of embodiments 65-76, further comprising one or more flame retardants, e.g., in an amount up to 20 wt %, up to 10 wt %, or up to 5 wt %.

Embodiment 78 is directed to the thermal management fluid of any of embodiments 65-77, wherein the thermal management fluid has a flash point of at least 50° C., e.g., at least 60° C., at least 65° C., at least 70° C., or at least 75° C., measured in accordance with ASTM D93.

Embodiment 79 is directed to the thermal management fluid of any of embodiments 65-77, wherein the thermal management fluid has a flash point of at least 90° C., e.g., at least 95° C., at least 100° C. or at least 105° C., measured in accordance with ASTM D93.

Embodiment 80 is directed to the thermal management fluid of any of embodiments 65-79, having a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

Embodiment 81 is directed to the thermal management fluid of any of embodiments 65-80, having a kinematic viscosity at 40° C. in the range of 2 to 20 cSt, e.g., in the range of 2 to 15 cSt, or 3 to 20 cSt, or 3 to 15 cSt, or 5 to 20 cSt, as measured in accordance with ASTM D455.

Embodiment 82 is directed to the thermal management fluid of any of embodiments 65-80, having a kinematic viscosity at 40° C. in the range of 2 to 10 cSt, e.g., 2 to 8 cSt, or 2 to 6 cSt, or 3 to 10 cSt, or 3 to 8 cSt, or 3 to 6 cSt, or 5 to 10 cSt, or 5 to 8 cSt, or 5 to 6 cSt, or 6 to 10 cSt, or 8 to 10 cSt, as measured in accordance with ASTM D455.

Embodiment 83 is directed to the thermal management fluid of any of embodiments 65-80, having a kinematic viscosity at 40° C. in the range of 2 to 5 cSt, or 2 to 4 cSt, or 2 to 3 cSt, or 3 to 5 cSt, or 3 to 4 cSt, or 4 to 5 cSt, as measured in accordance with ASTM D455.

Embodiment 84 is directed to the thermal management fluid of any of embodiments 65-80, having a dynamic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

Embodiment 85 is directed to the thermal management fluid of any of embodiments 65-84, having a dielectric constant of at least 1.5, e.g., at least 1.75, or at least 2.0, or at least 2.25, as measured at 25° C.

Embodiment 86 is directed to the thermal management fluid of any of embodiments 65-84, having a dielectric constant in the range of 1.5 to 10, or 1.8 to 10, or 1.5 to 2.8, or 1.8 to 2.8.

Embodiment 87 is directed to the thermal management fluid of any of embodiments 65-86, having a density of no more than 2.0 g/cm³ at 25° C. (e.g., no more than 1.8, or no more than 1.6 g/cm³ at 25° C.).

Embodiment 88 is directed to the thermal management fluid of any of embodiments 65-87, having a thermal conductivity in the range of 0.05 W/m·K to 1 W/m·K at 25° C.

Embodiment 89 is directed to the thermal management fluid of any of embodiments 65-88, having a specific heat capacity of at least 1 J/g·K (e.g., at least 1.2 J/g·K, or at least 1.5 J/g·K at 25° C.).

Embodiment 90 is directed to the thermal management fluid of any of embodiments 65-89, having a coefficient of thermal expansion of no more than 1100×10⁻⁶/K (e.g., no more than 1050×10⁻⁶/K, or no more than 1000×10⁻⁶/K).

Embodiment 91 is directed to the thermal management fluid of any of embodiments 65-91, wherein the thermal management fluid has a flash point of at least 50° C., e.g., at least 60° C., at least 65° C., at least 70° C., or at least 75° C., measured in accordance with ASTM D93 and has a kinematic viscosity at 25° C. in the range of 0.1 to 10 cSt, e.g., 0.2 to 8 cSt, or 0.2 to 6 cSt, or 0.3 to 10 cSt, or 0.3 to 8 cSt, or 0.3 to 6 cSt, or 0.5 to 10 cSt, or 0.5 to 8 cSt, or 0.5 to 6 cSt, or 0.5 to 5.5 cSt, as measured in accordance with ASTM D455.

Embodiment 92 is directed to the thermal management fluid of any of embodiments 65-91, wherein the thermal management fluid has a flash point of at least 50° C., e.g., at least 60° C., at least 65° C., at least 70° C., or at least 75° C., measured in accordance with ASTM D93 and has a kinematic viscosity at 40° C. in the range of 2 to 20 cSt, e.g., in the range of 2 to 15 cSt, or 3 to 20 cSt, or 3 to 15 cSt, or 5 to 20 cSt, as measured in accordance with ASTM D455.

Embodiment 93 is a method comprising:
contacting a thermal management fluid of embodiments 65-92 with a surface having a temperature of at least 25° C., the surface being in substantial thermal communication with a heat source; and
absorbing thermal energy in the thermal management fluid from the heat source through the surface.

Embodiment 94 is directed to the method according to embodiment 93, wherein the surface has a temperature of at least 30° C., e.g., at least 40° C.

Embodiment 95 is directed to the method according to embodiment 93, wherein the surface has a temperature in the range of 25° C. to 150° C., e.g., 25° C. to 100° C., or 25° C. to 90° C., or 25° C. to 85° C., or 25° C. to 80° C., or 25° C. to 75° C., or 25° C. to 70° C.

Embodiment 96 is directed to the method according to embodiment 93, wherein the surface has a temperature in the range of 30° C. to 150° C., e.g., 30° C. to 100° C., or 30° C. to 90° C., or 30° C. to 85° C., or 30° C. to 80° C., or 30° C. to 75° C., or 30° C. to 70° C.

Embodiment 97 is directed to the method according to embodiment 93, wherein the surface has a temperature in the range of 40° C. to 150° C., e.g., 50° C. to 150° C., or 60° C. to 150° C., or 70° C. to 150° C., or 80° C. to 150° C., or 90° C. to 150° C., or 100° C. to 150° C., or 110° C. to 150° C.

Embodiment 98 is directed to the method according to any of embodiments 93-97, wherein the thermal management fluid is a stationary (i.e., not circulating) body of fluid.

Embodiment 99 is directed to the method according to any of embodiments 93-97, wherein the contacting is performed by circulating the thermal management fluid over the surface.

Embodiment 100 is directed to the method according to any of embodiments 93-97, wherein the contacting is performed by circulating the thermal management fluid between a heat exchanger and the surface.

Embodiment 101 provides the method according to any of embodiments 93-100, wherein the thermal energy is absorbed at least in part by vaporizing one or more of the compounds as the thermal management fluid is heated through the boiling point(s) of the one or more halocarbons.

Embodiment 102 provides the method according to embodiment 101, further comprising condensing the one or more vaporized compounds and returning them to the thermal management fluid.

Embodiment 103 is directed to the method according to any of embodiments 93-101, wherein the heat source is an operating electrical component.

Embodiment 104 is directed to the method according to any of embodiments 93-101, wherein the heat source is a battery pack, a capacitor, inverter, electrical cabling, a fuel cell, a motor, a computer, or high power charging equipment.

Embodiment 105 is directed to the method according to any of embodiments 93-101, wherein the heat source is an electrochemical cell.

Embodiment 106 is directed to the method of embodiment 105, wherein the electrochemical cell is selected from solid state electrochemical cells, lithium-sulfur electrochemical cells, lithium iron phosphate electrochemical cells, lithium-ion polymer electrochemical cells, sodium-ion electrochemical cells, aluminum-ion cells, lead-acid cells, and magnesium-ion cells.

Embodiment 107 is directed to the method according to any of embodiments 93-101, wherein the surface is an internal surface of a conduit in substantial thermal communication with the heat source.

Embodiment 108 is directed to the method according to embodiment 107, wherein the conduit passes through a housing that surrounds the electrical component.

Embodiment 109 is directed to a battery system comprising:
a housing;
one or more electrochemical cells disposed in the housing;
a fluid path extending in the housing and in substantial thermal communication with the one or more electrochemical cells; and
a thermal management fluid of any of embodiments 65-92 disposed in the fluid path.

Embodiment 110 is directed to the battery system of embodiment 109, wherein the electrochemical cells are lithium-ion electrochemical cells.

Embodiment 111 is directed to the battery system of embodiment 109, wherein the electrochemical cells are solid state electrochemical cells, lithium-sulfur electrochemical cells, lithium iron phosphate electrochemical cells, lithium-ion polymer electrochemical cells, sodium-ion electrochemical cells, aluminum-ion cells, lead-acid cells, or magnesium-ion cells.

Embodiment 112 is directed to an electric vehicle comprising the battery system of any of embodiments 109-111.

Embodiment 113 is directed to a thermal management circuit comprising:
a fluid path extending around and/or through a heat source;
a thermal management fluid of any of embodiments 65-92, disposed in and configured to circulate in the fluid path and to absorb thermal energy produced by the heat source,
wherein the fluid is disposed in the fluid path, the heat exchanger, the pump and the connecting duct.

Embodiment 114 is directed to a data center hardware unit comprising the thermal management circuit of embodiment 113.

Embodiment 115 is directed to a high performance computer comprising the thermal management circuit of embodiment 113.

Embodiment 116 is directed to a method for preparing the compound of any of embodiments 1-64, the method comprising:
contacting a compound of formula (II)

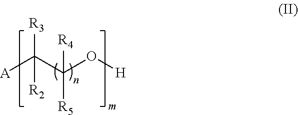

wherein
   m is an integer 1, 2, or 3;
   n is an integer in the range of 1-16;
   A is selected from H, OH and —O—R₆;
   each R₂ and R₃ is independently H or C₁-C₆ alkyl, and each R₄ and R₅ is independently H or C₁-C₆ alkyl, or
   each R₂ and R₄ is H or C₁-C₆ alkyl, and R₃ and R₅ together with the carbons to which they are bound come together to form a C₅-C₇ cycloalkyl; and
   R₆ is a C₁-C₁₂alkyl;
with CH₂═CF(CF₃) in the presence of a base to obtain a first compound of formula (I).

Embodiment 117 is directed to the method for preparing the compound of embodiment 116, wherein A is OH.

Embodiment 118 is directed to the method for preparing the compound of embodiment 116, wherein A is —O—R₆.

Embodiment 119 is directed to a method for preparing the compound of any of embodiments 116-118, further comprising hydrogenating the first compound of formula (I) to provide a second compound of formula (I).

Embodiment 120 is directed to a method for preparing the compound of any of embodiments 116-119, wherein the base is tBuOK.

Embodiment 121 is directed to a method for preparing the compound of any of embodiments 116-119, wherein the base is triethylamine.

The particulars shown herein are by way of example and for purposes of illustrative discussion of various embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparatus, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following embodiments and claims) are to be construed to cover both the singular and the plural, unless otherwise indicated herein or clearly contradicted by context.

The term “alkyl” as used herein, means a straight or branched chain hydrocarbon containing from 1 to 12 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

All percentages, ratios and proportions herein are by weight, unless otherwise specified.

Notwithstanding that the numerical ranges and parameters setting forth the broad scope of the disclosure are approximations, the numerical values set forth in the specific examples are reported as precisely as possible. Any numerical value, however, inherently contains various errors necessarily resulting from the standard deviation found in their respective testing measurements.

Groupings of alternative elements or embodiments of the disclosure are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of various aspects of the disclosure are described herein, including the best mode known to the inventors for carrying out the methods described herein. Of course, variations on these described embodiments will become apparent to those of ordinary skill in the art upon reading the foregoing description. The skilled artisan will employ such variations as appropriate, and as such the methods of the disclosure can be practiced otherwise than specifically described herein. Accordingly, the scope of the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

In closing, it is to be understood that the various embodiments herein are illustrative of the methods of the disclosures. Other modifications that may be employed are within the scope of the disclosure. Thus, by way of example, but not of limitation, alternative configurations of the methods may be utilized in accordance with the teachings herein. Accordingly, the methods of the present disclosure are not limited to that precisely as shown and described.

We claim:

1. A compound of formula (I): 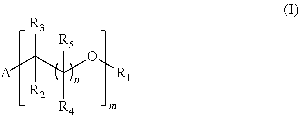
wherein
m is an integer 1, 2, or 3;
n is an integer in the range of 1-16;
A is H or —O—R₆;
R₁ is —C(═CH₂)—CF₃, or —CH(CH₃)—CF₃;
each of R₂ and R₃ is independently H or C₁-C₆ alkyl, and each of R₄ and R₅ is independently H or C₁-C₆ alkyl, or
each of R₂ and R₄ is H or C₁-C₆ alkyl, and R₃ and R₅ together with the carbons to which they are bound come together to form an C₅-C₇ cycloalkyl; and
R₆ is C₁-C₁₂ alkyl, —C(═CH₂)—CF₃, or —CH(CH₃)—CF₃.

2. The compound of claim 1, wherein R₆ is a branched C₆-C₁₂ alkyl.

3. The compound of claim 1, wherein each of R₂, R₃, R₄ and R₅ is independently selected from H, methyl, and ethyl.

4. The compound of claim 1, wherein each R₂ is H and each R₃ is H or C₁-C₆ alkyl (such as C₁-C₄ alkyl or C₁-C₂ alkyl); and each R₄ is H and each R₅ is H or C₁-C₆ alkyl (such as C₁-C₄ alkyl or C₁-C₂ alkyl).

5. The compound of claim 1, wherein R₂ is H, R₃ is H, R₄ is H and each R₅ is H or C₁-C₆ alkyl (such as C₁-C₄ alkyl or C₁-C₂ alkyl).

6. The compound of claim 1, having the formula 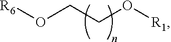
in which n is an integer in the range of 1-12.

7. The compound of claim 1, wherein m is 1.

8. The compound of claim 1, having a total number of carbon atoms from 5 to 30.

9. The compound of claim 1, selected from:

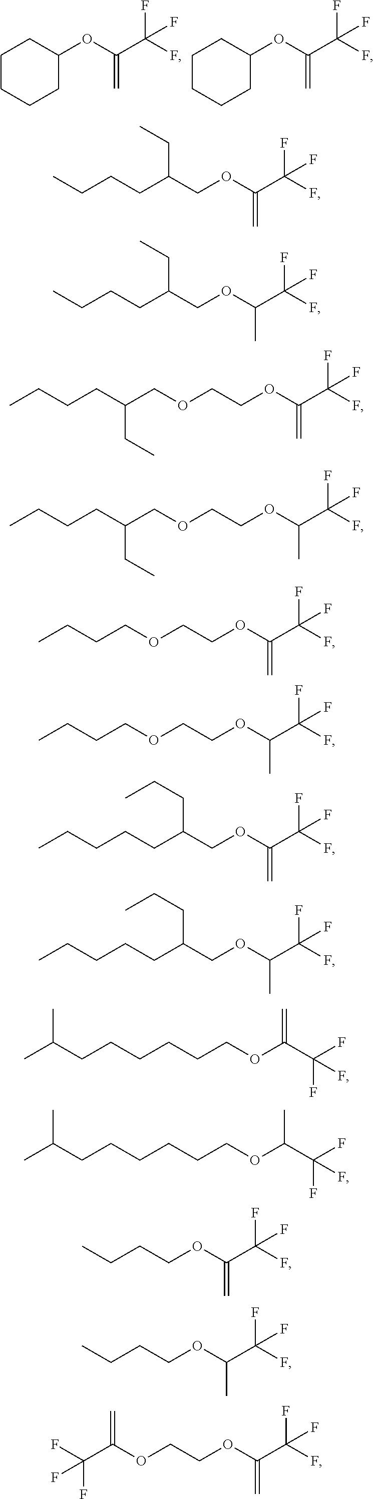

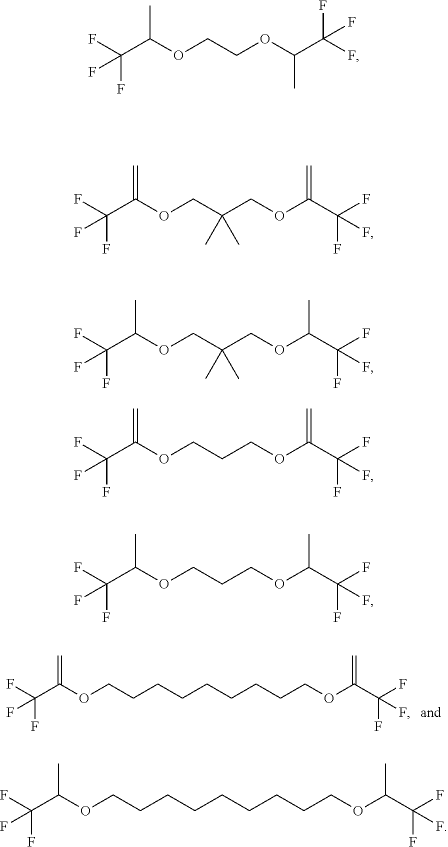

10. A thermal management fluid comprising one or more compounds according to any of claims 1-9, for example, present in an amount in the range of 50 wt % to 100 wt %, based on the total weight of the thermal management fluid.

11. The thermal management fluid of claim 10, wherein the thermal management fluid has a flash point of at least 50° C., measured in accordance with ASTM D93.

12. The thermal management fluid of claim 10, wherein the thermal management fluid has a kinematic viscosity at 25° C. in the range of 0.1 to 10 cSt, as measured in accordance with ASTM D455.

13. A method comprising:
contacting a thermal management fluid of claims 10-12 with a surface having a temperature of at least 25° C., the surface being in substantial thermal communication with a heat source; and
absorbing thermal energy in the thermal management fluid from the heat source through the surface.

14. The method according to claim 13, wherein the heat source is an operating electrical component, e.g., a battery pack, a capacitor, inverter, electrical cabling, a fuel cell, a motor, a computer, or high power charging equipment.

15. A battery system comprising:
a housing;
one or more electrochemical cells disposed in the housing;
a fluid path extending in the housing and in substantial thermal communication with the one or more electrochemical cells; and
a thermal management fluid of any of claims 10-12 disposed in the fluid path.

16. A thermal management circuit comprising:
a fluid path extending around and/or through a heat source;
a thermal management fluid of any of claims 10-12, disposed in and configured to circulate in the fluid path and to absorb thermal energy produced by the heat source,
wherein the fluid is disposed in the fluid path, the heat exchanger, the pump and the connecting duct.

17. A method of preparing a thermal management fluid comprising:
contacting a compound of formula (II) 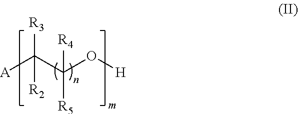
wherein
m is an integer 1, 2, or 3;
n is an integer in the range of 1-16;
A is selected from H, OH, or —O—R₆;
each R₂ and R₃ is independently H or C₁-C₆ alkyl, and each R₄ and R₅ is independently H or C₁-C₆ alkyl, or
each R₂ and R₄ is H or C₁-C₆ alkyl, and R₃ and R₅ together with the carbons to which they are bound come together to form a C₅-C₇ cycloalkyl; and
R₆ is a C₁-C₁₂alkyl;
with CH₂═CF(CF₃) in the presence of a base to provide a first compound of formula (I) in which R₁ is —C(═CH₂)—CF₃.

18. The method of claim 17, further hydrogenating the first compound of formula (I) to provide a second compound of formula (I) in which R₁ is —C(CH₃)—CF₃.
